SEQUENCE LISTING (30 sequences)

SEQ ID NO: 1 - protein, 557 aa, Artificial Sequence
  amino acid sequence of the extracellular
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACD

SEQ ID NO: 2 - protein, 557 aa, Artificial Sequence
  amino acid sequence of the extracellular
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACD

SEQ ID NO: 3 - protein, 556 aa, Artificial Sequence
  amino acid sequence of the extracellular
MALPVTALLLPLALLLHAARPQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACD

SEQ ID NO: 4 - protein, 577 aa, Artificial Sequence
  amino acid sequence of the extracellular
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVGGGGSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACD

SEQ ID NO: 5 - protein, 587 aa, Artificial Sequence
  amino acid sequence of the extracellular
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVGGGGSGGGGSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACD

SEQ ID NO: 6 - DNA, 1671 bp, Artificial Sequence
  nucleic acid molecule encoding the extracellular region
atggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggaggaggaggctccggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 7 - DNA, 1671 bp, Artificial Sequence
  nucleic acid molecule encoding the extracellular region
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggaggcggtgggtcgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggaggaggaggctccggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 8 - DNA, 1668 bp, Artificial Sequence
  nucleic acid molecule encoding the extracellular region
atggccttaccagtgaccgccttgctcctgccgctggccttgctgctccacgccgccaggccgcagatccagctggtgcagtctggcccagagctgaagaagcccggcgagaccgtgaagatcagctgcaaggcctccggctacaccttcacagactatagcatcaactgggtgaagagggcccctggcaagggcctgaagtggatgggctggatcaataccgagacacgcgagccagcctacgcctatgacttccggggcagattcgccttttccctggagacctctgccagcacagcctacctgcagatcaacaatctgaagtacgaggataccgccacatatttttgcgccctggactacagctatgccatggattattggggccagggcacctccgtgacagtgagctccggaggaggaggctccggcggcggaggctctggcggcggcggcagcgacatcgtgctgacccagtccccagcctctctggccatgtccctgggcaagcgggccacaatctcttgtagagcctccgagtctgtgagcgtgatcggcgcccacctgatccactggtaccagcagaagcctggccagccccctaagctgctgatctatctggccagcaacctggagaccggagtgccagcacggttctccggctctggcagcggcacagactttaccctgacaatcgatcctgtggaggaggacgatgtggccatctactcttgtctgcagagcaggatcttcccacgcacctttggcggcggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 9 - DNA, 1731 bp, Artificial Sequence
  nucleic acid molecule encoding the extracellular region
atggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggcggaggaggctctggcggaggaggctctggaggaggaggcagcggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggtggtgggtcgggcggtggtgggtcgggcggaggaggctctggaggaggaggcagcggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 10 - DNA, 1761 bp, Artificial Sequence
  nucleic acid molecule encoding the extracellular region
atggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggcggaggaggctctggcggtggtgggtcgggcggaggaggctctggaggaggaggcagcggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggtggtgggtcgggcggtggtgggtcgggcggtggtgggtcgggcggaggaggctctggaggaggaggcagcggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 11 - DNA, 72 bp, Artificial Sequence
  nucleic acid molecule encoding the transmembrane region
atctacatctgggcgcccttggccgggacttgtggggtccttctcctgtcactggttatcaccctttactgc

SEQ ID NO: 12 - DNA, 465 bp, Artificial Sequence
  nucleic acid molecule encoding the intracellular region
aaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 13 - DNA, 2221 bp, Artificial Sequence
  nucleic acid molecule encoding the chimeric antigen receptor
ggatcccgccaccatggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggaggaggaggctccggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - DNA, 2221 bp, Artificial Sequence
  nucleic acid molecule encoding the chimeric antigen receptor
ggatcccgccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggaggcggtgggtcgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggaggaggaggctccggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 15 - DNA, 2211 bp, Artificial Sequence
  nucleic acid molecule encoding the chimeric antigen receptor
gccaccatggccttaccagtgaccgccttgctcctgccgctggccttgctgctccacgccgccaggccgcagatccagctggtgcagtctggcccagagctgaagaagcccggcgagaccgtgaagatcagctgcaaggcctccggctacaccttcacagactatagcatcaactgggtgaagagggcccctggcaagggcctgaagtggatgggctggatcaataccgagacacgcgagccagcctacgcctatgacttccggggcagattcgccttttccctggagacctctgccagcacagcctacctgcagatcaacaatctgaagtacgaggataccgccacatatttttgcgccctggactacagctatgccatggattattggggccagggcacctccgtgacagtgagctccggaggaggaggctccggcggcggaggctctggcggcggcggcagcgacatcgtgctgacccagtccccagcctctctggccatgtccctgggcaagcgggccacaatctcttgtagagcctccgagtctgtgagcgtgatcggcgcccacctgatccactggtaccagcagaagcctggccagccccctaagctgctgatctatctggccagcaacctggagaccggagtgccagcacggttctccggctctggcagcggcacagactttaccctgacaatcgatcctgtggaggaggacgatgtggccatctactcttgtctgcagagcaggatcttcccacgcacctttggcggcggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - DNA, 2265 bp, Artificial Sequence
  nucleic acid molecule encoding the chimeric antigen receptor
atggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggcggaggaggctctggcggaggaggctctggaggaggaggcagcggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggtggtgggtcgggcggtggtgggtcgggcggaggaggctctggaggaggaggcagcggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 17 - DNA, 2295 bp, Artificial Sequence
  nucleic acid molecule encoding the chimeric antigen receptor
atggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggcggaggaggctctggcggtggtgggtcgggcggaggaggctctggaggaggaggcagcggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggtggtgggtcgggcggtggtgggtcgggcggtggtgggtcgggcggaggaggctctggaggaggaggcagcggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - DNA, 3403 bp, Artificial Sequence
  nucleic acid molecule carried by lentivirus
cgtgaggctccggtgcccgtcagtgggcagagcgcacatcgcccacagtccccgagaagttggggggaggggtcggcaattgacccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctcgccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagggatcccgccaccatggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggaggaggaggctccggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 19 - DNA, 3403 bp, Artificial Sequence
  nucleic acid molecule carried by lentivirus
cgtgaggctccggtgcccgtcagtgggcagagcgcacatcgcccacagtccccgagaagttggggggaggggtcggcaattgacccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctcgccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagggatcccgccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggaggcggtgggtcgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggaggaggaggctccggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - DNA, 3414 bp, Artificial Sequence
  nucleic acid molecule carried by lentivirus
cgtgaggctccggtgcccgtcagtgggcagagcgcacatcgcccacagtccccgagaagttggggggaggggtcggcaattgacccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctcgccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagggatcccgccaccatggccttaccagtgaccgccttgctcctgccgctggccttgctgctccacgccgccaggccgcagatccagctggtgcagtctggcccagagctgaagaagcccggcgagaccgtgaagatcagctgcaaggcctccggctacaccttcacagactatagcatcaactgggtgaagagggcccctggcaagggcctgaagtggatgggctggatcaataccgagacacgcgagccagcctacgcctatgacttccggggcagattcgccttttccctggagacctctgccagcacagcctacctgcagatcaacaatctgaagtacgaggataccgccacatatttttgcgccctggactacagctatgccatggattattggggccagggcacctccgtgacagtgagctccggaggaggaggctccggcggcggaggctctggcggcggcggcagcgacatcgtgctgacccagtccccagcctctctggccatgtccctgggcaagcgggccacaatctcttgtagagcctccgagtctgtgagcgtgatcggcgcccacctgatccactggtaccagcagaagcctggccagccccctaagctgctgatctatctggccagcaacctggagaccggagtgccagcacggttctccggctctggcagcggcacagactttaccctgacaatcgatcctgtggaggaggacgatgtggccatctactcttgtctgcagagcaggatcttcccacgcacctttggcggcggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatgaggccggcc

SEQ ID NO: 21 - DNA, 2274 bp, Artificial Sequence
  nucleic acid molecule carried by lentivirus
gccaccatggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggcggaggaggctctggcggaggaggctctggaggaggaggcagcggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggtggtgggtcgggcggtggtgggtcgggcggaggaggctctggaggaggaggcagcggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 22 - DNA, 2304 bp, Artificial Sequence
  nucleic acid molecule carried by lentivirus
gccaccatggccttaccagtgaccgccttgctcctgccgctggccttgctgctccacgccgccaggccgcaggtgcagctgcagcagagcggaccaggactggtgaagccatctcagaccctgagcctgacatgcgccatctctggcgatagcgtgagctccaactctgccgcctggaattggatcagacagtctccaagcagaggactggagtggctgggaaggacctactatagatccaagtggtacaacgattatgccgtgtccgtgaagtctcggatcaccatcaaccctgacacatccaagaatcagttctctctgcagctgaatagcgtgaccccagaggacacagccgtgtactattgcgcccgggaggtgaccggcgacctggaggatgcctttgacatctggggccagggcaccatggtgacagtgggcggaggaggctctggcggtggtgggtcgggcggaggaggctctggaggaggaggcagcggcggaggaggctctggaggaggaggcagcgacatccagatgacccagtccccatctagcctgtccgcctctgtgggcgacagggtgaccatcacatgtcgcgcctcccagacaatctggtcttacctgaactggtatcagcagagacccggcaaggcccctaatctgctgatctacgcagcctcctctctgcagagcggagtgccaagcaggttctccggccgcggctccggcaccgacttcaccctgacaatcagctccctgcaggccgaggacttcgccacatactattgtcagcagagctattccatccctcagacctttggccagggcacaaagctggagatcaagggaggcggtgggtcggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggcggtggtgggtcgggcggtggtgggtcgggcggtggtgggtcgggcggaggaggctctggaggaggaggcagcggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 23 - protein, 5 aa, Artificial Sequence
  Linker 1
GGGGS

SEQ ID NO: 24 - protein, 18 aa, Artificial Sequence
  Linker 6
GSTSGSGKPGSGEGSTKG

SEQ ID NO: 25 - protein, 107 aa, Artificial Sequence
  Amino acid sequence of light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 26 - protein, 120 aa, Artificial Sequence
  Amino acid sequence of heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 27 - protein, 107 aa, Artificial Sequence
  Amino acid sequence of light chain variable region
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 28 - protein, 122 aa, Artificial Sequence
  Amino acid sequence of heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTV

SEQ ID NO: 29 - protein, 111 aa, Artificial Sequence
  Amino acid sequence of light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 30 - protein, 117 aa, Artificial Sequence
  Amino acid sequence of heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

(19) United States
(12) Patent Application Publication
(10) Pub. No.: US 2023/0364136 A1
(43) Pub. Date: Nov. 16, 2023

(54) T lymphocyte and applications thereof

(71) Applicant: FAPON BIOTHERAPY INC., Shenzhen, Guangdong (CN)

(72) Inventors: Xiaolong DU, Shenzhen, Guangdong (CN); Baolei WANG, Shenzhen, Guangdong (CN); Liang PENG, Shenzhen, Guangdong (CN); Lijun YE, Shenzhen, Guangdong (CN)

(21) Appl. No.: 18/014,753

(22) PCT Filed: Jul. 6, 2021
(86) PCT No.: PCT/CN2021/104799

§ 371 Date: Jan. 6, 2023

(30) Foreign Application Priority Data
Jul. 7, 2020 (CN) 202010647746.7

(51) Int. Cl. A61K 35/17; C07K 16/28; C12N 15/86; A61P 35/02

(52) U.S. Cl. CPC A61K 35/17; C07K 16/2803; C07K 16/2878; C12N 15/86; A61P 35/02

(57) ABSTRACT

Provided is a T lymphocyte. The T lymphocyte expresses a chimeric antigen receptor, and includes an extracellular region. The extracellular region includes a first single-chain antibody, a second single-chain antibody, a first linker peptide, and a CD8 hinge region. The first linker peptide is arranged between the first single-chain antibody and the second single-chain antibody. The first single-chain antibody includes a first heavy chain variable region, a first light chain variable region, and a second linker peptide. The second linker peptide is arranged between the first heavy chain variable region and the first light chain variable region. The second single-chain antibody includes a second heavy chain variable region, a second light chain variable region, and a third linker peptide. The third linker peptide is arranged between the second heavy chain variable region and the second light chain variable region. The first linker peptide has a repeated amino acid sequence of GGGGS, and the second linker peptide and the third linker peptide independently have 2-6 repeated amino acid sequences of GGGGS, respectively.

### CROSS-REFERENCE TO RELATED APPLICATION

The present application is a National Stage of International Patent Application No. PCT/CN2021/104799, filed on Jul. 6, 2021,and claims priority to and interest of patent application No. 202010647746.7 filed to the China National Intellectual Property Administration on Jul. 7, 2020, the disclosure of which is hereby incorporated by reference in its entirety.

### SEQUENCE LISTING

The instant application contains a sequence listing which has submitted electronically in ASCII format and is hereby incorporated by reference in its entirety. Said ASCII copy is named PN199121_SEQ_LIST.txt and is 80,775 bytes in size. The sequence listing contains 30 sequences, in which the SEQ ID NOs:1-22are identical in substance to the sequences disclosed in the PCT application, and SEQ ID NOs:23-30 are newly added from the specification of the PCT application, and includes no new matter.

### TECHNICAL FIELD

The present disclosure relates to the field of biopharmaceuticals, specifically, the present disclosure relates to a T lymphocyte and applications thereof, and more specifically, the present disclosure relates to a T lymphocyte, a lentivirus, a transgenic lymphocyte, a construct, a method for preparing the T lymphocyte or the transgenic lymphocyte, a therapeutic composition for treating a cancer, and a method for improving lymphocyte activity.

### BACKGROUND

A hematological tumor is one of the top ten high incidence malignant tumors in China, and accounts for the sixth in the incidence rate of tumors. Especially, acute lymphoblastic leukemia (ALL), which mostly occurs in teenagers, is the malignant tumor with the highest incidence rate and mortality among people under 35 years old, herein acute B-lymphoblastic leukemia (B-ALL) is the most common.

Some researches show that about 90% of R/R B-ALL patients get complete remission (CR) after receiving CAR-T19 (a lymphocyte expressing a chimeric antigen receptor (CAR) targeting CD19). Although the initial response rate is very high, many patients relapse, and more than 30% of the relapsed patients treated with Blinatumomab and more than 60% of the relapsed patients treated with CAR-T19 lose a CD19 antigen target, so that a CD19 specific immunotherapy is unable to identify malignant cells. These phenomena also explain the advantages and disadvantages of the antigen specific immunotherapy.

There are two main forms of the relapse in the process of CAR-T19 treatment, the first is that the patient loses CAR-T19 at an early stage and relapses, and the other is that CAR-T19 is still present but CD19-leukemia appears, this type also appears after receiving the Blinatumomab.

Therefore, a CAR-T cell still needs to be further developed and improved.

### SUMMARY

The present disclosure aims to at least solve one of technical problems in related technologies to a certain extent.

In a first aspect of the present disclosure, the present disclosure provides a T lymphocyte. According to an embodiment of the present disclosure, the T lymphocyte expresses a chimeric antigen receptor, the chimeric antigen receptor includes: an extracellular region, herein the extracellular region includes a first single-chain antibody, a second single-chain antibody, a first linker peptide and a CD8 hinge region, the first single-chain antibody specifically recognizes a first antigen, the second single-chain antibody specifically recognizes a second antigen, the first linker peptide is arranged between the first single-chain antibody and the second single-chain antibody, the first single-chain antibody includes a first heavy chain variable region, a first light chain variable region and a second linker peptide, the second linker peptide is arranged between the first heavy chain variable region and the first light chain variable region, the second single-chain antibody includes a second heavy chain variable region, a second light chain variable region and a third linker peptide, the third linker peptide is arranged between the second heavy chain variable region and the second light chain variable region, the first linker peptide has a repeated amino acid sequence of GGGGS(SEQ ID NO:23), and the second linker peptide and the third linker peptide independently have 2˜6 repeated amino acid sequences of GGGGS respectively; a transmembrane region, herein the transmembrane region is connected with the extracellular region, the transmembrane region includes a transmembrane segment of CD8, and is embedded into a cell membrane of the T lymphocyte; and an intracellular region, herein the intracellular region is connected with the transmembrane region, and the intracellular region includes an intracellular segment of 4-1BB and a CD3 ζ chain. The T lymphocyte according to the embodiment of the present disclosure may specifically kill single/double positive tumor cells of the first antigen and the second antigen, and has a longer and more powerful tumor cell killing and eliminating effect in vivo compared with an existing technology.

According to an embodiment of the present disclosure, the above T lymphocyte may further include at least one of the following additional technical features.

According to an embodiment of the present disclosure, the first single-chain antibody is a CD19 single-chain antibody, the second single-chain antibody is a CD22 single-chain antibody, the first antigen is CD19, the second antigen is CD22, the first heavy chain variable region is a CD19 heavy chain variable region, the first light chain variable region is a CD19 light chain variable region, the second heavy chain variable region is a CD22 heavy chain variable region, and the second light chain variable region is a CD22 light chain variable region. The T lymphocyte according to the embodiment of the present disclosure may specifically kill the single/double positive tumor cells of CD19 and CD22.

According to an embodiment of the present disclosure, the first single-chain antibody is a CD19 single-chain antibody, the second single-chain antibody is a B cell maturation antigen (BCMA) single-chain antibody, the first antigen is CD19, the second antigen is BCMA, the first heavy chain variable region is a CD19 heavy chain variable region, the first light chain variable region is a CD19 light chain variable region, the second heavy chain variable region is a BCMA heavy chain variable region, and the second light chain variable region is a BCMA light chain variable region.

According to an embodiment of the present disclosure, the N terminal of the first linker peptide is connected with the C terminal of the second single-chain antibody, the C terminal of the first linker peptide is connected with the N terminal of the first single-chain antibody, and the C terminal of the first single-chain antibody is connected with the N terminal of the CD8 hinge region. It is found by the inventor that in the above connecting order of the second single-chain antibody, the first linker peptide and the first single-chain antibody, the secretion of cytokines, such as IL-2 and IFN-γ, is lower, and in vivo treatment of the T lymphocyte according to the embodiment of the present disclosure is safer.

According to an embodiment of the present disclosure, the N terminal of the first linker peptide is connected with the C terminal of the CD22 single-chain antibody, the C terminal of the first linker peptide is connected with the N terminal of the CD19 single-chain antibody, and the C terminal of the CD19 single-chain antibody is connected with the N terminal of the CD8 hinge region. The above T lymphocyte according to the embodiment of the present disclosure may specifically kill the single/double positive tumor cells of CD19 and CD22, and the cytokine secretion in vivo is lower. Compared with the existing technology, it has the longer and more powerful effect of killing and eliminating blood tumor cells in vivo.

According to an embodiment of the present disclosure, the N terminal of the first linker peptide is connected with the C terminal of the CD19 single-chain antibody, the C terminal of the first linker peptide is connected with the N terminal of the CD22 single-chain antibody, and the C terminal of the CD22 single-chain antibody is connected with the N terminal of the CD8 hinge region. The above T lymphocyte according to the embodiment of the present disclosure may specifically kill the single/double positive tumor cells of CD19 and CD22, and has the longer and more powerful effect of killing and eliminating the blood tumor cells in vivo compared with the existing technology.

According to an embodiment of the present disclosure, the N terminal of the third linker peptide is connected with the C terminal of the BCMA heavy chain variable region, the C terminal of the third linker peptide is connected with the N terminal of the BCMA light chain variable region, the C terminal of the BCMA light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region. The above T lymphocyte according to the embodiment of the present disclosure may specifically kill the single/double positive tumor cells of BCMA and CD19, and has the longer and more powerful effect of killing and eliminating the tumor cells in vivo compared with the existing technology.

According to an embodiment of the present disclosure, the N terminal of the third linker peptide is connected with the C terminal of the CD22 heavy chain variable region, the C terminal of the third linker peptide is connected with the N terminal of the CD22 light chain variable region, the C terminal of the CD22 light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region. The above T lymphocyte according to the embodiment of the present disclosure may specifically kill the single/double positive tumor cells of CD19 and CD22, and the cytokine secretion in vivo is lower. Compared with the existing technology, it has the longer and more powerful effect of killing and eliminating the blood tumor cells in vivo.

According to an embodiment of the present disclosure, the N terminal of the second linker peptide is connected with the C terminal of the CD19 light chain variable region, the C terminal of the second linker peptide is connected with the N terminal of the CD19 heavy chain variable region, the C terminal of the CD19 heavy chain variable region is connected with the N terminal of the first linker peptide, the C terminal of the first linker peptide is connected with the N terminal of the CD22 heavy chain variable region, the N terminal of the third linker peptide is connected with the C terminal of the CD22 heavy chain variable region, and the C terminal of the third linker peptide is connected with the N terminal of the CD22 light chain variable region, and the C terminal of the CD22 light chain variable region is connected with the N terminal of the CD8 hinge region. The above T lymphocyte according to the embodiment of the present disclosure may specifically kill the single/double positive tumor cells of CD19 and CD22, and has the longer and more powerful effect of killing and eliminating the blood tumor cells in vivo compared with the existing technology.

According to an embodiment of the present disclosure, the second linker peptide and the third linker peptide independently have 3˜5 repeated amino acid sequences of GGGGS respectively.

According to an embodiment of the present disclosure, the second linker peptide and the third linker peptide have 5 repeated amino acid sequences of GGGGS respectively. It is found by the inventor that while the second linker peptide and the third linker peptide have 5 repeated GGGGS respectively, the killing effect of the T lymphocyte according to the embodiment of the present disclosure is stronger, and after being introduced into a body, the amount of the cytokines secreted by the body is lower, and the safety is higher.

According to an embodiment of the present disclosure, the extracellular region has an amino acid sequence shown in SEQ ID NO: 1˜5.

(SEQ ID NO: 1)
MALPVTALLLPLALLLHAARPQVQLQQSGPGLVKPSQTLSLTCAISGDSVSSNSAAWNWIRQS
PSRGLEWLGRTYYRSKWYNDYAVSVKSRITINPDTSKNQFSLQLNSVTPEDTAVYYCAREVTGDL
EDAFDIWGQGTMVTVGGGGSGGGGSGGGGSDIQMTQSPSSLSASVGDRVTITCRASQTIWSYL
NWYQQRPGKAPNLLIYAASSLQSGVPSRFSGRGSGTDFTLTISSLQAEDFATYYCQQSYSIPQTFG
QGTKLEIKGGGGSDIQMTQTTSSLSASLGDRVTISCRASQDISKYLNWYQQKPDGTVKLLIYHTSR
LHSGVPSRFSGSGSGTDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLEWLGVIWGSETTYYN
SALKSRLTIIKDNSKSQVFLKMNSLQTDDTAIYYCAKHYYYGGSYAMDYWGQGTSVTVSSTTTPAP
RPPTPAPTIASQPLSLRPEACRPAAGGAVHTRGLDFACD.
(SEQ ID NO: 2)
MALPVTALLLPLALLLHAARPDIQMTQTTSSLSASLGDRVTISCRASQDISKYLNWYQQKPDGT
VKLLIYHTSRLHSGVPSRFSGSGSGTDYSLTISNLEQEDIATYFCQQGNTLPYTFGGGTKLEITGGG
GSGGGGSGGGGSEVKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYYGGSYAMDYWGQGTSV
TVSSGGGGSQVQLQQSGPGLVKPSQTLSLTCAISGDSVSSNSAAWNWIRQSPSRGLEWLGRTYY
RSKWYNDYAVSVKSRITINPDTSKNQFSLQLNSVTPEDTAVYYCAREVTGDLEDAFDIWGQGTMV
TVGGGGSGGGGSGGGGSDIQMTQSPSSLSASVGDRVTITCRASQTIWSYLNWYQQRPGKAPNL
LIYAASSLQSGVPSRFSGRGSGTDFTLTISSLQAEDFATYYCQQSYSIPQTFGQGTKLEIKTTTPAP
RPPTPAPTIASQPLSLRPEACRPAAGGAVHTRGLDFACD.
(SEQ ID NO: 3)
MALPVTALLLPLALLLHAARPQIQLVQSGPELKKPGETVKISCKASGYTFTDYSINWWVKRAPGK
GLKWMGWINTETREPAYAYDFRGRFAFSLETSASTAYLQINNLKYEDTATYFCALDYSYAMDYWG
QGTSVTVSSGGGGSGGGGSGGGGSDIVLTQSPASLAMSLGKRATISCRASESVSVIGAHLIHWYQ
QKPGQPPKLLIYLASNLETGVPARFSGSGSGTDFTLTIDPVEEDDVAIYSCLQSRIFPRTFGGGTKL
EIKGGGGSDIQMTQTTSSLSASLGDRVTISCRASQDISKYLNWYQQKPDGTVKLLIYHTSRLHSGV
PSRFSGSGSGTDYSLTISNLEQEDIATYFCQQGNTLPYTFGGGTKLEITGGGGSGGGGSGGGGSE
VKLQESGPGLVAPSQSLSVTCTVSGVSLPDYGVSWIRQPPRKGLEWLGVIWGSETTYYNSALKSR
LTIIKDNSKSQVFLKMNSLQTDDTAIYYCAKHYYYGGSYAMDYWGQGTSVTVSSTTTPAPRPPTPA
PTIASQPLSLRPEACRPAAGGAVHTRGLDFACD.
(SEQ ID NO: 4)
MALPVTALLLPLALLLHAARPQVQLQQSGPGLVKPSQTLSLTCAISGDSVSSNSAAWNWIRQS
PSRGLEWLGRTYYRSKWYNDYAVSVKSRITINPDTSKNQFSLQLNSVTPEDTAVYYCAREVTGDL
EDAFDIWGQGTMVTVGGGGSGGGGSGGGGSGGGGSGGGGSDIQMTQSPSSLSASVGDRVTIT
CRASQTIWSYLNWYQQRPGKAPNLLIYAASSLQSGVPSRFSGRGSGTDFTLTISSLQAEDFATYYC
QQSYSIPQTFGQGTKLEIKGGGGSDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGTKLEITG
GGGSGGGGSGGGGSGGGGSGGGGSEVKLQESGPGLVAPSQSLSVTCTVSGVSLPDYGVSWIR
QPPRKGLEWLGVIWGSETTYYNSALKSRLTIIKDNSKSQVFLKMNSLQTDDTAIYYCAKHYYYGGS
YAMDYWGQGTSVTVSSTTTPAPRPPTPAPTIASQPLSLRPEACRPAAGGAVHTRGLDFACD.
(SEQ ID NO: 5)
MALPVTALLLPLALLLHAARPQVQLQQSGPGLVKPSQTLSLTCAISGDSVSSNSAAWNWIRQS
PSRGLEWLGRTYYRSKWYNDYAVSVKSRITINPDTSKNQFSLQLNSVTPEDTAVYYCAREVTGDL
EDAFDIWGQGTMVTVGGGGSGGGGSGGGGSGGGGSGGGGSGGGGSDIQMTQSPSSLSASVG
DRVTITCRASQTIWSYLNWYQQRPGKAPNLLIYAASSLQSGVPSRFSGRGSGTDFTLTISSLQAED
FATYYCQQSYSIPQTFGQGTKLEIKGGGGSDIQMTQTTSSLSASLGDRVTISCRASQDISKYLNWY
QQKPDGTVKLLIYHTSRLHSGVPSRFSGSGSGTDYSLTISNLEQEDIATYFCQQGNTLPYTFGGGT
KLEITGGGGSGGGGSGGGGSGGGGSGGGGSGGGGSEVKLQESGPGLVAPSQSLSVTCTVSGV
SLPDYGVSWIRQPPRKGLEWLGVIWGSETTYYNSALKSRLTIIKDNSKSQVFLKMNSLQTDDTAIYY
CAKHYYYGGSYAMDYWGQGTSVTVSSTTTPAPRPPTPAPTIASQPLSLRPEACRPAAGGAVHTR
GLDFACD.

Herein, the amino acid sequence shown in SEQ ID NO: 1 is a sequence of an extracellular region of a chimeric antigen receptor expressed by Car-pCDHF32 (the molecular structure is shown in FIG. 1), the amino acid sequence shown in SEQ ID NO: 2 is a sequence of an extracellular region of a chimeric antigen receptor expressed by Car-pCDHF34 (the molecular structure is shown in FIG. 2), the amino acid sequence shown in SEQ ID NO: 3 is a sequence of an extracellular region of a chimeric antigen receptor expressed by Car-pCDHF31 (the molecular structure is shown in FIG. 15), the amino acid sequence shown in SEQ ID NO: 4 is a sequence of an extracellular region of a chimeric antigen receptor expressed by Car-pCDHF58 (except that the second and third linker peptides have 5 repeated GGGGS, the rest of the structure is the same as the molecular structure of Car-pCDHF32), and the amino acid sequence shown in SEQ ID NO: 5 is a sequence of an extracellular region of a chimeric antigen receptor expressed by Car-pCDH F59 (except that the second and third linker peptides have 6 repeated GGGGS, the rest of the structure is the same as the molecular structure of Car-pCDHF32).

In a second aspect of the present disclosure, the present disclosure provides a lentivirus. According to an embodiment of the present disclosure, the lentivirus carries a nucleic acid molecule encoding a chimeric antigen receptor, and the chimeric antigen receptor includes: an extracellular region, herein the extracellular region includes a first single-chain antibody, a second single-chain antibody, a first linker peptide and a CD8 hinge region, the first single-chain antibody specifically recognizes a first antigen, the second single-chain antibody specifically recognizes a second antigen, the first linker peptide is arranged between the first single-chain antibody and the second single-chain antibody, the first single-chain antibody includes a first heavy chain variable region, a first light chain variable region and a second linker peptide, the second linker peptide is arranged between the first heavy chain variable region and the first light chain variable region, the second single-chain antibody includes a second heavy chain variable region, a second light chain variable region and a third linker peptide, the third linker peptide is arranged between the second heavy chain variable region and the second light chain variable region, the first linker peptide has a repeated amino acid sequence of GGGGS, and the second linker peptide and the third linker peptide independently have 2˜6 repeated amino acid sequences of GGGGS respectively; a transmembrane region, herein the transmembrane region is connected with the extracellular region, the transmembrane region includes a transmembrane segment of CD8, and is embedded into a cell membrane of the T lymphocyte; and an intracellular region, herein the intracellular region is connected with the transmembrane region, and the intracellular region includes an intracellular segment of 4-1 BB and a CD3 ζ chain. The lentivirus according to the embodiment of the present disclosure is introduced into a receptor cell-T lymphocyte, and the previously described T lymphocyte may be obtained. The T lymphocyte obtained may specifically kill single/double positive tumor cells of the first antigen and the second antigen, and has the longer and more powerful effect of killing and eliminating the tumor cells in vivo compared with the existing technology.

According to an embodiment of the present disclosure, the above lentivirus may further include at least one of the following additional technical features.

According to an embodiment of the disclosure, the first single-chain antibody is a CD19 single-chain antibody, the second single-chain antibody is a CD22 single-chain antibody, the first antigen is CD19, the second antigen is CD22, the first heavy chain variable region is a CD19 heavy chain variable region, the first light chain variable region is a CD19 light chain variable region, the second heavy chain variable region is a CD22 heavy chain variable region, and the second light chain variable region is a CD22 light chain variable region.

According to an embodiment of the present disclosure, the first single-chain antibody is a CD19 single-chain antibody, the second single-chain antibody is a BCMA single-chain antibody, the first antigen is CD19, the second antigen is BCMA, the first heavy chain variable region is a CD19 heavy chain variable region, the first light chain variable region is a CD19 light chain variable region, the second heavy chain variable region is a BCMA heavy chain variable region, and the second light chain variable region is a BCMA light chain variable region.

According to an embodiment of the present disclosure, the N terminal of the third linker peptide is connected with the C terminal of the CD22 heavy chain variable region, the C terminal of the third linker peptide is connected with the N terminal of the CD22 light chain variable region, the C terminal of the CD22 light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region. The above lentivirus according to the embodiment of the present disclosure is introduced into a T lymphocyte, the T lymphocyte obtained may specifically kill the single/double positive tumor cells of CD19 and CD22, and the cytokine secretion in vivo is lower. Compared with the existing technology, it has the longer and more powerful effect of killing and eliminating the blood tumor cells in vivo.

According to an embodiment of the present disclosure, the N terminal of the second linker peptide is connected with the C terminal of the CD19 light chain variable region, the C terminal of the second linker peptide is connected with the N terminal of the CD19 heavy chain variable region, the C terminal of the CD19 heavy chain variable region is connected with the N terminal of the first linker peptide, the C terminal of the first linker peptide is connected with the N terminal of the CD22 heavy chain variable region, the N terminal of the third linker peptide is connected with the C terminal of the CD22 heavy chain variable region, and the C terminal of the third linker peptide is connected with the N terminal of the CD22 light chain variable region, and the C terminal of the CD22 light chain variable region is connected with the N terminal of the CD8 hinge region. The above lentivirus according to the embodiment of the present disclosure is introduced into a T lymphocyte, the T lymphocyte obtained may specifically kill the single/double positive tumor cells of CD19 and CD22, and has the longer and more powerful effect of killing and eliminating the blood tumor cells in vivo compared with the existing technology.

According to an embodiment of the present disclosure, the N terminal of the third linker peptide is connected with the C terminal of the BCMA heavy chain variable region, the C terminal of the third linker peptide is connected with the N terminal of the BCMA light chain variable region, the C terminal of the BCMA light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region. The above lentivirus according to the embodiment of the present disclosure is introduced into a T lymphocyte, the T lymphocyte obtained may specifically kill the single/double positive tumor cells of CD19 and BCMA, and has the longer and more powerful effect of killing and eliminating the tumor cells in vivo compared with the existing technology.

According to an embodiment of the present disclosure, the nucleic acid molecule encoding the extracellular region has any one of nucleotide sequences shown in SEQ ID NO: 6˜10.

(SEQ ID NO: 6)
ATGGCCTTACCAGTGACCGCCTTGCTCCTGCCGCTGGCCTTGCTGCTCCACGCCGCCAG
GCCGCAGGTGCAGCTGCAGCAGAGCGGACCAGGACTGGTGAAGCCATCTCAGACCCTGAGC
CTGACATGCGCCATCTCTGGCGATAGCGTGAGCTCCAACTCTGCCGCCTGGAATTGGATCAGA
CAGTCTCCAAGCAGAGGACTGGAGTGGCTGGGAAGGACCTACTATAGATCCAAGTGGTACAA
CGATTATGCCGTGTCCGTGAAGTCTCGGATCACCATCAACCCTGACACATCCAAGAATCAGTTC
TCTCTGCAGCTGAATAGCGTGACCCCAGAGGACACAGCCGTGTACTATTGCGCCCGGGAGGT
GACCGGCGACCTGGAGGATGCCTTTGACATCTGGGGCCAGGGCACCATGGTGACAGTGGGA
GGAGGAGGCTCCGGCGGAGGAGGCTCTGGAGGAGGAGGCAGCGACATCCAGATGACCCAG
TCCCCATCTAGCCTGTCCGCCTCTGTGGGCGACAGGGTGACCATCACATGTCGCGCCTCCCA
GACAATCTGGTCTTACCTGAACTGGTATCAGCAGAGACCCGGCAAGGCCCCTAATCTGCTGAT
CTACGCAGCCTCCTCTCTGCAGAGCGGAGTGCCAAGCAGGTTCTCCGGCCGCGGCTCCGGC
ACCGACTTCACCCTGACAATCAGCTCCCTGCAGGCCGAGGACTTCGCCACATACTATTGTCAG
CAGAGCTATTCCATCCCTCAGACCTTTGGCCAGGGCACAAAGCTGGAGATCAAGGGAGGCGG
TGGGTCGGACATCCAGATGACACAGACTACATCCTCCCTGTCTGCCTCTCTGGGAGACAGAGT
CACCATCAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATCAAGGTT
CAGTGGCAGTGGGTCTGGAACAGATTATTCTCTCACCATTAGCAACCTGGAGCAAGAAGATATT
GCCACTTACTTTTGCCAACAGGGTAATACGCTTCCGTACACGTTCGGAGGGGGGACCAAGCTG
GAGATCACAGGTGGCGGTGGCTCGGGCGGTGGTGGGTCGGGTGGCGGCGGATCTGAGGTG
AAACTGCAGGAGTCAGGACCTGGCCTGGTGGCGCCCTCACAGAGCCTGTCCGTCACATGCA
CTGTCTCAGGGGTCTCATTACCCGACTATGGTGTAAGCTGGATTCGCCAGCCTCCACGAAAGG
GTCTGGAGTGGCTGGGAGTAATATGGGGTAGTGAAACCACATACTATAATTCAGCTCTCAAATC
CAGACTGACCATCATCAAGGACAACTCCAAGAGCCAAGTTTTCTTAAAAATGAACAGTCTGCAA
ACTGATGACACAGCCATTTACTACTGTGCCAAACATTATTACTACGGTGGTAGCTATGCTATGGA
CTACTGGGGCCAAGGAACCTCAGTCACCGTCTCCTCAACCACGACGCCAGCGCCGCGACCA
CCAACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGAT.
(SEQ ID NO: 7)
ATGGCCTTACCAGTGACCGCCTTGCTCCTGCCGCTGGCCTTGCTGCTCCACGCCGCCAG
GCCGGACATCCAGATGACACAGACTACATCCTCCCTGTCTGCCTCTCTGGGAGACAGAGTCA
CCATCAGTTGCAGGGCAAGTCAGGACATTAGTAAATATTTAAATTGGTATCAGCAGAAACCAGAT
GGAACTGTTAAACTCCTGATCTACCATACATCAAGATTACACTCAGGAGTCCCATCAAGGTTCA
GTGGCAGTGGGTCTGGAACAGATTATTCTCTCACCATTAGCAACCTGGAGCAAGAAGATATTGC
CACTTACTTTTGCCAACAGGGTAATACGCTTCCGTACACGTTCGGAGGGGGGACCAAGCTGGA
GATCACAGGTGGCGGTGGCTCGGGCGGTGGTGGGTCGGGTGGCGGCGGATCTGAGGTGAA
ACTGCAGGAGTCAGGACCTGGCCTGGTGGCGCCCTCACAGAGCCTGTCCGTCACATGCACT
GTCTCAGGGGTCTCATTACCCGACTATGGTGTAAGCTGGATTCGCCAGCCTCCACGAAAGGGT
CTGGAGTGGCTGGGAGTAATATGGGGTAGTGAAACCACATACTATAATTCAGCTCTCAAATCCA
GACTGACCATCATCAAGGACAACTCCAAGAGCCAAGTTTTCTTAAAAATGAACAGTCTGCAAAC
TGATGACACAGCCATTTACTACTGTGCCAAACATTATTACTACGGTGGTAGCTATGCTATGGACT
ACTGGGGCCAAGGAACCTCAGTCACCGTCTCCTCAGGAGGCGGTGGGTCGCAGGTGCAGCT
GCAGCAGAGCGGACCAGGACTGGTGAAGCCATCTCAGACCCTGAGCCTGACATGCGCCATCT
CTGGCGATAGCGTGAGCTCCAACTCTGCCGCCTGGAATTGGATCAGACAGTCTCCAAGCAGA
GGACTGGAGTGGCTGGGAAGGACCTACTATAGATCCAAGTGGTACAACGATTATGCCGTGTCC
GTGAAGTCTCGGATCACCATCAACCCTGACACATCCAAGAATCAGTTCTCTCTGCAGCTGAATA
GCGTGACCCCAGAGGACACAGCCGTGTACTATTGCGCCCGGGAGGTGACCGGCGACCTGGA
GGATGCCTTTGACATCTGGGGCCAGGGCACCATGGTGACAGTGGGAGGAGGAGGCTCCGGC
GGAGGAGGCTCTGGAGGAGGAGGCAGCGACATCCAGATGACCCAGTCCCCATCTAGCCTGT
CCGCCTCTGTGGGCGACAGGGTGACCATCACATGTCGCGCCTCCCAGACAATCTGGTCTTAC
CTGAACTGGTATCAGCAGAGACCCGGCAAGGCCCCTAATCTGCTGATCTACGCAGCCTCCTCT
CTGCAGAGCGGAGTGCCAAGCAGGTTCTCCGGCCGCGGCTCCGGCACCGACTTCACCCTGA
CAATCAGCTCCCTGCAGGCCGAGGACTTCGCCACATACTATTGTCAGCAGAGCTATTCCATCC
CTCAGACCTTTGGCCAGGGCACAAAGCTGGAGATCAAGACCACGACGCCAGCGCCGCGACC
ACCAACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGAT.
(SEQ ID NO: 8)
ATGGCCTTACCAGTGACCGCCTTGCTCCTGCCGCTGGCCTTGCTGCTCCACGCCGCCAG
GCCGCAGATCCAGCTGGTGCAGTCTGGCCCAGAGCTGAAGAAGCCCGGCGAGACCGTGAAG
ATCAGCTGCAAGGCCTCCGGCTACACCTTCACAGACTATAGCATCAACTGGGTGAAGAGGGCC
CCTGGCAAGGGCCTGAAGTGGATGGGCTGGATCAATACCGAGACACGCGAGCCAGCCTACG
CCTATGACTTCCGGGGCAGATTCGCCTTTTCCCTGGAGACCTCTGCCAGCACAGCCTACCTGC
AGATCAACAATCTGAAGTACGAGGATACCGCCACATATTTTTGCGCCCTGGACTACAGCTATGC
CATGGATTATTGGGGCCAGGGCACCTCCGTGACAGTGAGCTCCGGAGGAGGAGGCTCCGGC
GGCGGAGGCTCTGGCGGCGGCGGCAGCGACATCGTGCTGACCCAGTCCCCAGCCTCTCTG
GCCATGTCCCTGGGCAAGCGGGCCACAATCTCTTGTAGAGCCTCCGAGTCTGTGAGCGTGAT
CGGCGCCCACCTGATCCACTGGTACCAGCAGAAGCCTGGCCAGCCCCCTAAGCTGCTGATCT
ATCTGGCCAGCAACCTGGAGACCGGAGTGCCAGCACGGTTCTCCGGCTCTGGCAGCGGCAC
AGACTTTACCCTGACAATCGATCCTGTGGAGGAGGACGATGTGGCCATCTACTCTTGTCTGCA
GAGCAGGATCTTCCCACGCACCTTTGGCGGCGGCACAAAGCTGGAGATCAAGGGAGGCGGT
GGGTCGGACATCCAGATGACACAGACTACATCCTCCCTGTCTGCCTCTCTGGGAGACAGAGT
CACCATCAGTTGCAGGGCAAGTCAGGACATTAGTAAATATTTAAATTGGTATCAGCAGAAACCA
GATGGAACTGTTAAACTCCTGATCTACCATACATCAAGATTACACTCAGGAGTCCCATCAAGGTT
CAGTGGCAGTGGGTCTGGAACAGATTATTCTCTCACCATTAGCAACCTGGAGCAAGAAGATATT
GCCACTTACTTTTGCCAACAGGGTAATACGCTTCCGTACACGTTCGGAGGGGGGACCAAGCTG
GAGATCACAGGTGGCGGTGGCTCGGGCGGTGGTGGGTCGGGTGGCGGCGGATCTGAGGTG
AAACTGCAGGAGTCAGGACCTGGCCTGGTGGCGCCCTCACAGAGCCTGTCCGTCACATGCA
CTGTCTCAGGGGTCTCATTACCCGACTATGGTGTAAGCTGGATTCGCCAGCCTCCACGAAAGG
GTCTGGAGTGGCTGGGAGTAATATGGGGTAGTGAAACCACATACTATAATTCAGCTCTCAAATC
CAGACTGACCATCATCAAGGACAACTCCAAGAGCCAAGTTTTCTTAAAAATGAACAGTCTGCAA
ACTGATGACACAGCCATTTACTACTGTGCCAAACATTATTACTACGGTGGTAGCTATGCTATGGA
CTACTGGGGCCAAGGAACCTCAGTCACCGTCTCCTCAACCACGACGCCAGCGCCGCGACCA
CCAACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGAT.
(SEQ ID NO: 9)
ATGGCCTTACCAGTGACCGCCTTGCTCCTGCCGCTGGCCTTGCTGCTCCACGCCGCCAG
GCCGCAGGTGCAGCTGCAGCAGAGCGGACCAGGACTGGTGAAGCCATCTCAGACCCTGAGC
CTGACATGCGCCATCTCTGGCGATAGCGTGAGCTCCAACTCTGCCGCCTGGAATTGGATCAGA
CAGTCTCCAAGCAGAGGACTGGAGTGGCTGGGAAGGACCTACTATAGATCCAAGTGGTACAA
CGATTATGCCGTGTCCGTGAAGTCTCGGATCACCATCAACCCTGACACATCCAAGAATCAGTTC
TCTCTGCAGCTGAATAGCGTGACCCCAGAGGACACAGCCGTGTACTATTGCGCCCGGGAGGT
GACCGGCGACCTGGAGGATGCCTTTGACATCTGGGGCCAGGGCACCATGGTGACAGTGGGC
GGAGGAGGCTCTGGCGGAGGAGGCTCTGGAGGAGGAGGCAGCGGCGGAGGAGGCTCTGG
AGGAGGAGGCAGCGACATCCAGATGACCCAGTCCCCATCTAGCCTGTCCGCCTCTGTGGGCG
ACAGGGTGACCATCACATGTCGCGCCTCCCAGACAATCTGGTCTTACCTGAACTGGTATCAGC
AGAGACCCGGCAAGGCCCCTAATCTGCTGATCTACGCAGCCTCCTCTCTGCAGAGCGGAGTG
CCAAGCAGGTTCTCCGGCCGCGGCTCCGGCACCGACTTCACCCTGACAATCAGCTCCCTGCA
GGCCGAGGACTTCGCCACATACTATTGTCAGCAGAGCTATTCCATCCCTCAGACCTTTGGCCA
GGGCACAAAGCTGGAGATCAAGGGAGGCGGTGGGTCGGACATCCAGATGACACAGACTACAT
CCTCCCTGTCTGCCTCTCTGGGAGACAGAGTCACCATCAGTTGCAGGGCAAGTCAGGACATT
AGTAAATATTTAAATTGGTATCAGCAGAAACCAGATGGAACTGTTAAACTCCTGATCTACCATACA
TCAAGATTACACTCAGGAGTCCCATCAAGGTTCAGTGGCAGTGGGTCTGGAACAGATTATTCT
CTCACCATTAGCAACCTGGAGCAAGAAGATATTGCCACTTACTTTTGCCAACAGGGTAATACGC
TTCCGTACACGTTCGGAGGGGGGACCAAGCTGGAGATCACAGGCGGTGGTGGGTCGGGCGG
TGGTGGGTCGGGCGGAGGAGGCTCTGGAGGAGGAGGCAGCGGTGGCGGCGGATCTGAGGT
GAAACTGCAGGAGTCAGGACCTGGCCTGGTGGCGCCCTCACAGAGCCTGTCCGTCACATGC
ACTGTCTCAGGGGTCTCATTACCCGACTATGGTGTAAGCTGGATTCGCCAGCCTCCACGAAAG
GGTCTGGAGTGGCTGGGAGTAATATGGGGTAGTGAAACCACATACTATAATTCAGCTCTCAAAT
CCAGACTGACCATCATCAAGGACAACTCCAAGAGCCAAGTTTTCTTAAAAATGAACAGTCTGCA
AACTGATGACACAGCCATTTACTACTGTGCCAAACATTATTACTACGGTGGTAGCTATGCTATGG
ACTACTGGGGCCAAGGAACCTCAGTCACCGTCTCCTCAACCACGACGCCAGCGCCGCGACC
ACCAACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGAT.
(SEQ ID NO: 10)
ATGGCCTTACCAGTGACCGCCTTGCTCCTGCCGCTGGCCTTGCTGCTCCACGCCGCCAG
GCCGCAGGTGCAGCTGCAGCAGAGCGGACCAGGACTGGTGAAGCCATCTCAGACCCTGAGC
CTGACATGCGCCATCTCTGGCGATAGCGTGAGCTCCAACTCTGCCGCCTGGAATTGGATCAGA
CAGTCTCCAAGCAGAGGACTGGAGTGGCTGGGAAGGACCTACTATAGATCCAAGTGGTACAA
CGATTATGCCGTGTCCGTGAAGTCTCGGATCACCATCAACCCTGACACATCCAAGAATCAGTTC
TCTCTGCAGCTGAATAGCGTGACCCCAGAGGACACAGCCGTGTACTATTGCGCCCGGGAGGT
GACCGGCGACCTGGAGGATGCCTTTGACATCTGGGGCCAGGGCACCATGGTGACAGTGGGC
GGAGGAGGCTCTGGCGGTGGTGGGTCGGGCGGAGGAGGCTCTGGAGGAGGAGGCAGCGG
CGGAGGAGGCTCTGGAGGAGGAGGCAGCGACATCCAGATGACCCAGTCCCCATCTAGCCTG
TCCGCCTCTGTGGGCGACAGGGTGACCATCACATGTCGCGCCTCCCAGACAATCTGGTCTTA
CCTGAACTGGTATCAGCAGAGACCCGGCAAGGCCCCTAATCTGCTGATCTACGCAGCCTCCTC
TCTGCAGAGCGGAGTGCCAAGCAGGTTCTCCGGCCGCGGCTCCGGCACCGACTTCACCCTG
ACAATCAGCTCCCTGCAGGCCGAGGACTTCGCCACATACTATTGTCAGCAGAGCTATTCCATC
CCTCAGACCTTTGGCCAGGGCACAAAGCTGGAGATCAAGGGAGGCGGTGGGTCGGACATCC
AGATGACACAGACTACATCCTCCCTGTCTGCCTCTCTGGGAGACAGAGTCACCATCAGTTGCA
GGGCAAGTCAGGACATTAGTAAATATTTAAATTGGTATCAGCAGAAACCAGATGGAACTGTTAAA
CTCCTGATCTACCATACATCAAGATTACACTCAGGAGTCCCATCAAGGTTCAGTGGCAGTGGGT
CTGGAACAGATTATTCTCTCACCATTAGCAACCTGGAGCAAGAAGATATTGCCACTTACTTTTGC
CAACAGGGTAATACGCTTCCGTACACGTTCGGAGGGGGGACCAAGCTGGAGATCACAGGCGG
TGGTGGGTCGGGCGGTGGTGGGTCGGGCGGTGGTGGGTCGGGCGGAGGAGGCTCTGGAG
GAGGAGGCAGCGGTGGCGGCGGATCTGAGGTGAAACTGCAGGAGTCAGGACCTGGCCTGG
TGGCGCCCTCACAGAGCCTGTCCGTCACATGCACTGTCTCAGGGGTCTCATTACCCGACTATG
GTGTAAGCTGGATTCGCCAGCCTCCACGAAAGGGTCTGGAGTGGCTGGGAGTAATATGGGGT
AGTGAAACCACATACTATAATTCAGCTCTCAAATCCAGACTGACCATCATCAAGGACAACTCCAA
GAGCCAAGTTTTCTTAAAAATGAACAGTCTGCAAACTGATGACACAGCCATTTACTACTGTGCC
AAACATTATTACTACGGTGGTAGCTATGCTATGGACTACTGGGGCCAAGGAACCTCAGTCACCG
TCTCCTCAACCACGACGCCAGCGCCGCGACCACCAACACCGGCGCCCACCATCGCGTCGCA
GCCCCTGTCCCTGCGCCCAGAGGCGTGCCGGCCAGCGGCGGGGGGCGCAGTGCACACGAG
GGGGCTGGACTTCGCCTGTGAT.

The nucleic acid molecule with the nucleotide sequence shown in SEQ ID NO: 6 encodes an extracellular region of a chimeric antigen receptor with the amino acid sequence shown in SEQ ID NO: 1; the nucleic acid molecule with the nucleotide sequence shown in SEQ ID NO: 7 encodes an extracellular region of a chimeric antigen receptor with the amino acid sequence shown in SEQ ID NO: 2; the nucleic acid molecule with the nucleotide sequence shown in SEQ ID NO: 8 encodes an extracellular region of a chimeric antigen receptor with the amino acid sequence shown in SEQ ID NO: 3; the nucleic acid molecule with the nucleotide sequence shown in SEQ ID NO: 9 encodes an extracellular region of a chimeric antigen receptor with the amino acid sequence shown in SEQ ID NO: 4; and the nucleic acid molecule with the nucleotide sequence shown in SEQ ID NO: 10 encodes an extracellular region of a chimeric antigen receptor with the amino acid sequence shown in SEQ ID NO: 5.

According to an embodiment of the present disclosure, the nucleic acid molecule encoding the transmembrane region has a nucleotide sequence shown in SEQ ID NO: 11.

(SEQ ID NO: 11)
ATCTACATCTGGGCGCCCTTGGCCGGGACTTGTGGGGTCCTTCTCCTGTCACTGGTTATC
ACCCTTTACTGC.

According to an embodiment of the present disclosure, the nucleic acid molecule encoding the intracellular region has a nucleotide sequence shown in SEQ ID NO: 12.

(SEQ ID NO: 12)
AAACGGGGCAGAAAGAAACTCCTGTATATATTCAAACAACCATTTATGAGACCAGTACAAAC
TACTCAAGAGGAAGATGGCTGTAGCTGCCGATTTCCAGAAGAAGAAGAAGGAGGATGTGAACT
GAGAGTGAAGTTCAGCAGGAGCGCAGACGCCCCCGCGTACAAGCAGGGCCAGAACCAGCTC
TATAACGAGCTCAATCTAGGACGAAGAGAGGAGTACGATGTTTTGGACAAGAGACGTGGCCGG
GACCCTGAGATGGGGGGAAAGCCGAGAAGGAAGAACCCTCAGGAAGGCCTGTACAATGAACT
GCAGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGACGC
CCTTCACATGCAGGCCCTGCCCCCTCGCTAA.

According to an embodiment of the present disclosure, the nucleic acid molecule encoding the chimeric antigen receptor has a nucleotide sequence shown in SEQ ID NO: 13˜17.

(SEQ ID NO: 13)
GGATCCCGCCACCATGGCCTTACCAGTGACCGCCTTGCTCCTGCCGCTGGCCTTGCTGC
TCCACGCCGCCAGGCCGCAGGTGCAGCTGCAGCAGAGCGGACCAGGACTGGTGAAGCCATC
TCAGACCCTGAGCCTGACATGCGCCATCTCTGGCGATAGCGTGAGCTCCAACTCTGCCGCCT
GGAATTGGATCAGACAGTCTCCAAGCAGAGGACTGGAGTGGCTGGGAAGGACCTACTATAGAT
CCAAGTGGTACAACGATTATGCCGTGTCCGTGAAGTCTCGGATCACCATCAACCCTGACACAT
CCAAGAATCAGTTCTCTCTGCAGCTGAATAGCGTGACCCCAGAGGACACAGCCGTGTACTATT
GCGCCCGGGAGGTGACCGGCGACCTGGAGGATGCCTTTGACATCTGGGGCCAGGGCACCAT
GGTGACAGTGGGAGGAGGAGGCTCCGGCGGAGGAGGCTCTGGAGGAGGAGGCAGCGACAT
CCAGATGACCCAGTCCCCATCTAGCCTGTCCGCCTCTGTGGGCGACAGGGTGACCATCACAT
GTCGCGCCTCCCAGACAATCTGGTCTTACCTGAACTGGTATCAGCAGAGACCCGGCAAGGCC
CCTAATCTGCTGATCTACGCAGCCTCCTCTCTGCAGAGCGGAGTGCCAAGCAGGTTCTCCGG
CCGCGGCTCCGGCACCGACTTCACCCTGACAATCAGCTCCCTGCAGGCCGAGGACTTCGCC
ACATACTATTGTCAGCAGAGCTATTCCATCCCTCAGACCTTTGGCCAGGGCACAAAGCTGGAG
ATCAAGGGAGGCGGTGGGTCGGACATCCAGATGACACAGACTACATCCTCCCTGTCTGCCTC
TCTGGGAGACAGAGTCACCATCAGTTGCAGGGCAAGTCAGGACATTAGTAAATATTTAAATTGG
TATCAGCAGAAACCAGATGGAACTGTTAAACTCCTGATCTACCATACATCAAGATTACACTCAGG
AGTCCCATCAAGGTTCAGTGGCAGTGGGTCTGGAACAGATTATTCTCTCACCATTAGCAACCT
GGAGCAAGAAGATATTGCCACTTACTTTTGCCAACAGGGTAATACGCTTCCGTACACGTTCGGA
GGGGGGACCAAGCTGGAGATCACAGGTGGCGGTGGCTCGGGCGGTGGTGGGTCGGGTGGC
GGCGGATCTGAGGTGAAACTGCAGGAGTCAGGACCTGGCCTGGTGGCGCCCTCACAGAGCC
TGTCCGTCACATGCACTGTCTCAGGGGTCTCATTACCCGACTATGGTGTAAGCTGGATTCGCC
AGCCTCCACGAAAGGGTCTGGAGTGGCTGGGAGTAATATGGGGTAGTGAAACCACATACTATA
ATTCAGCTCTCAAATCCAGACTGACCATCATCAAGGACAACTCCAAGAGCCAAGTTTTCTTAAA
AATGAACAGTCTGCAAACTGATGACACAGCCATTTACTACTGTGCCAAACATTATTACTACGGTG
GTAGCTATGCTATGGACTACTGGGGCCAAGGAACCTCAGTCACCGTCTCCTCAACCACGACGC
CAGCGCCGCGACCACCAACACCGGCGCCCACCATCGCGTCGCAGCCCCTGTCCCTGCGCCC
AGAGGCGTGCCGGCCAGCGGCGGGGGGCGCAGTGCACACGAGGGGGCTGGACTTCGCCT
GTGATATCTACATCTGGGCGCCCTTGGCCGGGACTTGTGGGGTCCTTCTCCTGTCACTGGTTA
TCACCCTTTACTGCAAACGGGGCAGAAAGAAACTCCTGTATATATTCAAACAACCATTTATGAGA
CCAGTACAAACTACTCAAGAGGAAGATGGCTGTAGCTGCCGATTTCCAGAAGAAGAAGAAGGA
GGATGTGAACTGAGAGTGAAGTTCAGCAGGAGCGCAGACGCCCCCGCGTACAAGCAGGGCC
AGAACCAGCTCTATAACGAGCTCAATCTAGGACGAAGAGAGGAGTACGATGTTTTGGACAAGA
GACGTGGCCGGGACCCTGAGATGGGGGGAAAGCCGAGAAGGAAGAACCCTCAGGAAGGCC
TGTACAATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCAAGGA
CACCTACGACGCCCTTCACATGCAGGCCCTGCCCCCTCGCTAA.
(SEQ ID NO: 14)
GGATCCCGCCACCATGGCCTTACCAGTGACCGCCTTGCTCCTGCCGCTGGCCTTGCTGC
TCCACGCCGCCAGGCCGGACATCCAGATGACACAGACTACATCCTCCCTGTCTGCCTCTCTG
GGAGACAGAGTCACCATCAGTTGCAGGGCAAGTCAGGACATTAGTAAATATTTAAATTGGTATC
AGCAGAAACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAACCTGGA
GCAAGAAGATATTGCCACTTACTTTTGCCAACAGGGTAATACGCTTCCGTACACGTTCGGAGGG
GGGACCAAGCTGGAGATCACAGGTGGCGGTGGCTCGGGCGGTGGTGGGTCGGGTGGCGGC
GGATCTGAGGTGAAACTGCAGGAGTCAGGACCTGGCCTGGTGGCGCCCTCACAGAGCCTGT
CCGTCACATGCACTGTCTCAGGGGTCTCATTACCCGACTATGGTGTAAGCTGGATTCGCCAGC
CTCCACGAAAGGGTCTGGAGTGGCTGGGAGTAATATGGGGTAGTGAAACCACATACTATAATTC
AGCTCTCAAATCCAGACTGACCATCATCAAGGACAACTCCAAGAGCCAAGTTTTCTTAAAAATG
AACAGTCTGCAAACTGATGACACAGCCATTTACTACTGTGCCAAACATTATTACTACGGTGGTA
GCTATGCTATGGACTACTGGGGCCAAGGAACCTCAGTCACCGTCTCCTCAGGAGGCGGTGGG
TCGCAGGTGCAGCTGCAGCAGAGCGGACCAGGACTGGTGAAGCCATCTCAGACCCTGAGCC
TGACATGCGCCATCTCTGGCGATAGCGTGAGCTCCAACTCTGCCGCCTGGAATTGGATCAGAC
AGTCTCCAAGCAGAGGACTGGAGTGGCTGGGAAGGACCTACTATAGATCCAAGTGGTACAAC
GATTATGCCGTGTCCGTGAAGTCTCGGATCACCATCAACCCTGACACATCCAAGAATCAGTTCT
CTCTGCAGCTGAATAGCGTGACCCCAGAGGACACAGCCGTGTACTATTGCGCCCGGGAGGTG
ACCGGCGACCTGGAGGATGCCTTTGACATCTGGGGCCAGGGCACCATGGTGACAGTGGGAG
GAGGAGGCTCCGGCGGAGGAGGCTCTGGAGGAGGAGGCAGCGACATCCAGATGACCCAGT
CCCCATCTAGCCTGTCCGCCTCTGTGGGCGACAGGGTGACCATCACATGTCGCGCCTCCCAG
ACAATCTGGTCTTACCTGAACTGGTATCAGCAGAGACCCGGCAAGGCCCCTAATCTGCTGATC
TACGCAGCCTCCTCTCTGCAGAGCGGAGTGCCAAGCAGGTTCTCCGGCCGCGGCTCCGGCA
CCGACTTCACCCTGACAATCAGCTCCCTGCAGGCCGAGGACTTCGCCACATACTATTGTCAGC
AGAGCTATTCCATCCCTCAGACCTTTGGCCAGGGCACAAAGCTGGAGATCAAGACCACGACG
CCAGCGCCGCGACCACCAACACCGGCGCCCACCATCGCGTCGCAGCCCCTGTCCCTGCGCC
CAGAGGCGTGCCGGCCAGCGGCGGGGGGCGCAGTGCACACGAGGGGGCTGGACTTCGCCT
GTGATATCTACATCTGGGCGCCCTTGGCCGGGACTTGTGGGGTCCTTCTCCTGTCACTGGTTA
TCACCCTTTACTGCAAACGGGGCAGAAAGAAACTCCTGTATATATTCAAACAACCATTTATGAGA
CCAGTACAAACTACTCAAGAGGAAGATGGCTGTAGCTGCCGATTTCCAGAAGAAGAAGAAGGA
GGATGTGAACTGAGAGTGAAGTTCAGCAGGAGCGCAGACGCCCCCGCGTACAAGCAGGGCC
AGAACCAGCTCTATAACGAGCTCAATCTAGGACGAAGAGAGGAGTACGATGTTTTGGACAAGA
GACGTGGCCGGGACCCTGAGATGGGGGGAAAGCCGAGAAGGAAGAACCCTCAGGAAGGCC
TGTACAATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCAAGGA
CACCTACGACGCCCTTCACATGCAGGCCCTGCCCCCTCGCTAA.
(SEQ ID NO: 15)
GCCACCATGGCCTTACCAGTGACCGCCTTGCTCCTGCCGCTGGCCTTGCTGCTCCACGC
CGCCAGGCCGCAGATCCAGCTGGTGCAGTCTGGCCCAGAGCTGAAGAAGCCCGGCGAGACC
GTGAAGATCAGCTGCAAGGCCTCCGGCTACACCTTCACAGACTATAGCATCAACTGGGTGAAG
AGGGCCCCTGGCAAGGGCCTGAAGTGGATGGGCTGGATCAATACCGAGACACGCGAGCCAG
CCTACGCCTATGACTTCCGGGGCAGATTCGCCTTTTCCCTGGAGACCTCTGCCAGCACAGCCT
ACCTGCAGATCAACAATCTGAAGTACGAGGATACCGCCACATATTTTTGCGCCCTGGACTACAG
CTATGCCATGGATTATTGGGGCCAGGGCACCTCCGTGACAGTGAGCTCCGGAGGAGGAGGCT
CCGGCGGCGGAGGCTCTGGCGGCGGCGGCAGCGACATCGTGCTGACCCAGTCCCCAGCCT
CTCTGGCCATGTCCCTGGGCAAGCGGGCCACAATCTCTTGTAGAGCCTCCGAGTCTGTGAGC
GTGATCGGCGCCCACCTGATCCACTGGTACCAGCAGAAGCCTGGCCAGCCCCCTAAGCTGCT
GATCTATCTGGCCAGCAACCTGGAGACCGGAGTGCCAGCACGGTTCTCCGGCTCTGGCAGC
GGCACAGACTTTACCCTGACAATCGATCCTGTGGAGGAGGACGATGTGGCCATCTACTCTTGT
CTGCAGAGCAGGATCTTCCCACGCACCTTTGGCGGCGGCACAAAGCTGGAGATCAAGGGAG
GCGGTGGGTCGGACATCCAGATGACACAGACTACATCCTCCCTGTCTGCCTCTCTGGGAGAC
AGAGTCACCATCAGTTGCAGGGCAAGTCAGGACATTAGTAAATATTTAAATTGGTATCAGCAGA
AACCAGATGGAACTGTTAAACTCCTGATCTACCATACATCAAGATTACACTCAGGAGTCCCATCA
AGGTTCAGTGGCAGTGGGTCTGGAACAGATTATTCTCTCACCATTAGCAACCTGGAGCAAGAA
GATATTGCCACTTACTTTTGCCAACAGGGTAATACGCTTCCGTACACGTTCGGAGGGGGGACC
AAGCTGGAGATCACAGGTGGCGGTGGCTCGGGCGGTGGTGGGTCGGGTGGCGGCGGATCT
GAGGTGAAACTGCAGGAGTCAGGACCTGGCCTGGTGGCGCCCTCACAGAGCCTGTCCGTCA
CATGCACTGTCTCAGGGGTCTCATTACCCGACTATGGTGTAAGCTGGATTCGCCAGCCTCCAC
GAAAGGGTCTGGAGTGGCTGGGAGTAATATGGGGTAGTGAAACCACATACTATAATTCAGCTCT
CAAATCCAGACTGACCATCATCAAGGACAACTCCAAGAGCCAAGTTTTCTTAAAAATGAACAGT
CTGCAAACTGATGACACAGCCATTTACTACTGTGCCAAACATTATTACTACGGTGGTAGCTATGC
TATGGACTACTGGGGCCAAGGAACCTCAGTCACCGTCTCCTCAACCACGACGCCAGCGCCGC
GACCACCAACACCGGCGCCCACCATCGCGTCGCAGCCCCTGTCCCTGCGCCCAGAGGCGTG
CCGGCCAGCGGCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTTATCACCCTTTAC
TGCAAACGGGGCAGAAAGAAACTCCTGTATATATTCAAACAACCATTTATGAGACCAGTACAAA
CTACTCAAGAGGAAGATGGCTGTAGCTGCCGATTTCCAGAAGAAGAAGAAGGAGGATGTGAAC
TGAGAGTGAAGTTCAGCAGGAGCGCAGACGCCCCCGCGTACAAGCAGGGCCAGAACCAGCT
CTATAACGAGCTCAATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCCTGTACAATGAA
CTGCAGAAAGATAAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGGACACCTACGACG
CCCTTCACATGCAGGCCCTGCCCCCTCGCTAA.
(SEQ ID NO: 16)
ATGGCCTTACCAGTGACCGCCTTGCTCCTGCCGCTGGCCTTGCTGCTCCACGCCGCCAG
GCCGCAGGTGCAGCTGCAGCAGAGCGGACCAGGACTGGTGAAGCCATCTCAGACCCTGAGC
CTGACATGCGCCATCTCTGGCGATAGCGTGAGCTCCAACTCTGCCGCCTGGAATTGGATCAGA
CAGTCTCCAAGCAGAGGACTGGAGTGGCTGGGAAGGACCTACTATAGATCCAAGTGGTACAA
CGATTATGCCGTGTCCGTGAAGTCTCGGATCACCATCAACCCTGACACATCCAAGAATCAGTTC
TCTCTGCAGCTGAATAGCGTGACCCCAGAGGACACAGCCGTGTACTATTGCGCCCGGGAGGT
GACCGGCGACCTGGAGGATGCCTTTGACATCTGGGGCCAGGGCACCATGGTGACAGTGGGC
GGAGGAGGCTCTGGCGGAGGAGGCTCTGGAGGAGGAGGCAGCGGCGGAGGAGGCTCTGG
AGGAGGAGGCAGCGACATCCAGATGACCCAGTCCCCATCTAGCCTGTCCGCCTCTGTGGGCG
ACAGGGTGACCATCACATGTCGCGCCTCCCAGACAATCTGGTCTTACCTGAACTGGTATCAGC
AGAGACCCGGCAAGGCCCCTAATCTGCTGATCTACGCAGCCTCCTCTCTGCAGAGCGGAGTG
CCAAGCAGGTTCTCCGGCCGCGGCTCCGGCACCGACTTCACCCTGACAATCAGCTCCCTGCA
GGCCGAGGACTTCGCCACATACTATTGTCAGCAGAGCTATTCCATCCCTCAGACCTTTGGCCA
GGGCACAAAGCTGGAGATCAAGGGAGGCGGTGGGTCGGACATCCAGATGACACAGACTACAT
CCTCCCTGTCTGCCTCTCTGGGAGACAGAGTCACCATCAGTTGCAGGGCAAGTCAGGACATT
AGTAAATATTTAAATTGGTATCAGCAGAAACCAGATGGAACTGTTAAACTCCTGATCTACCATACA
TCAAGATTACACTCAGGAGTCCCATCAAGGTTCAGTGGCAGTGGGTCTGGAACAGATTATTCT
CTCACCATTAGCAACCTGGAGCAAGAAGATATTGCCACTTACTTTTGCCAACAGGGTAATACGC
TTCCGTACACGTTCGGAGGGGGGACCAAGCTGGAGATCACAGGCGGTGGTGGGTCGGGCGG
TGGTGGGTCGGGCGGAGGAGGCTCTGGAGGAGGAGGCAGCGGTGGCGGCGGATCTGAGGT
GAAACTGCAGGAGTCAGGACCTGGCCTGGTGGCGCCCTCACAGAGCCTGTCCGTCACATGC
ACTGTCTCAGGGGTCTCATTACCCGACTATGGTGTAAGCTGGATTCGCCAGCCTCCACGAAAG
GGTCTGGAGTGGCTGGGAGTAATATGGGGTAGTGAAACCACATACTATAATTCAGCTCTCAAAT
CCAGACTGACCATCATCAAGGACAACTCCAAGAGCCAAGTTTTCTTAAAAATGAACAGTCTGCA
AACTGATGACACAGCCATTTACTACTGTGCCAAACATTATTACTACGGTGGTAGCTATGCTATGG
ACTACTGGGGCCAAGGAACCTCAGTCACCGTCTCCTCAACCACGACGCCAGCGCCGCGACC
ACCAACACCGGCGCCCACCATCGCGTCGCAGCCCCTGTCCCTGCGCCCAGAGGCGTGCCGG
CCAGCGGCGGGGGGCGCAGTGCACACGAGGGGGCTGGACTTCGCCTGTGATATCTACATCT
GGGCGCCCTTGGCCGGGACTTGTGGGGTCCTTCTCCTGTCACTGGTTATCACCCTTTACTGC
AAACGGGGCAGAAAGAAACTCCTGTATATATTCAAACAACCATTTATGAGACCAGTACAAACTAC
TCAAGAGGAAGATGGCTGTAGCTGCCGATTTCCAGAAGAAGAAGAAGGAGGATGTGAACTGA
GAGTGAAGTTCAGCAGGAGCGCAGACGCCCCCGCGTACAAGCAGGGCCAGAACCAGCTCTA
TAACGAGCTCAATCTAGGACGAAGAGAGGAGTACGATGTTTTGGACAAGAGACGTGGCCGGG
ACCCTGAGATGGGGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGG
GCAAGGGGCACGATGGCCTTTACCAGGGTCTCAGTACAGCCACCAAGGACACCTACGACGCC
CTTCACATGCAGGCCCTGCCCCCTCGC.
(SEQ ID NO: 17)
ATGGCCTTACCAGTGACCGCCTTGCTCCTGCCGCTGGCCTTGCTGCTCCACGCCGCCAG
GCCGCAGGTGCAGCTGCAGCAGAGCGGACCAGGACTGGTGAAGCCATCTCAGACCCTGAGC
CTGACATGCGCCATCTCTGGCGATAGCGTGAGCTCCAACTCTGCCGCCTGGAATTGGATCAGA
CAGTCTCCAAGCAGAGGACTGGAGTGGCTGGGAAGGACCTACTATAGATCCAAGTGGTACAA
CGATTATGCCGTGTCCGTGAAGTCTCGGATCACCATCAACCCTGACACATCCAAGAATCAGTTC
TCTCTGCAGCTGAATAGCGTGACCCCAGAGGACACAGCCGTGTACTATTGCGCCCGGGAGGT
GACCGGCGACCTGGAGGATGCCTTTGACATCTGGGGCCAGGGCACCATGGTGACAGTGGGC
GGAGGAGGCTCTGGCGGTGGTGGGTCGGGCGGAGGAGGCTCTGGAGGAGGAGGCAGCGG
CGGAGGAGGCTCTGGAGGAGGAGGCAGCGACATCCAGATGACCCAGTCCCCATCTAGCCTG
TCCGCCTCTGTGGGCGACAGGGTGACCATCACATGTCGCGCCTCCCAGACAATCTGGTCTTA
CCTGAACTGGTATCAGCAGAGACCCGGCAAGGCCCCTAATCTGCTGATCTACGCAGCCTCCTC
TCTGCAGAGCGGAGTGCCAAGCAGGTTCTCCGGCCGCGGCTCCGGCACCGACTTCACCCTG
ACAATCAGCTCCCTGCAGGCCGAGGACTTCGCCACATACTATTGTCAGCAGAGCTATTCCATC
CCTCAGACCTTTGGCCAGGGCACAAAGCTGGAGATCAAGGGAGGCGGTGGGTCGGACATCC
AGATGACACAGACTACATCCTCCCTGTCTGCCTCTCTGGGAGACAGAGTCACCATCAGTTGCA
GGGCAAGTCAGGACATTAGTAAATATTTAAATTGGTATCAGCAGAAACCAGATGGAACTGTTAAA
CTCCTGATCTACCATACATCAAGATTACACTCAGGAGTCCCATCAAGGTTCAGTGGCAGTGGGT
CTGGAACAGATTATTCTCTCACCATTAGCAACCTGGAGCAAGAAGATATTGCCACTTACTTTTGC
CAACAGGGTAATACGCTTCCGTACACGTTCGGAGGGGGGACCAAGCTGGAGATCACAGGCGG
TGGTGGGTCGGGCGGTGGTGGGTCGGGCGGTGGTGGGTCGGGCGGAGGAGGCTCTGGAG
GAGGAGGCAGCGGTGGCGGCGGATCTGAGGTGAAACTGCAGGAGTCAGGACCTGGCCTGG
TGGCGCCCTCACAGAGCCTGTCCGTCACATGCACTGTCTCAGGGGTCTCATTACCCGACTATG
GTGTAAGCTGGATTCGCCAGCCTCCACGAAAGGGTCTGGAGTGGCTGGGAGTAATATGGGGT
AGTGAAACCACATACTATAATTCAGCTCTCAAATCCAGACTGACCATCATCAAGGACAACTCCAA
GAGCCAAGTTTTCTTAAAAATGAACAGTCTGCAAACTGATGACACAGCCATTTACTACTGTGCC
AAACATTATTACTACGGTGGTAGCTATGCTATGGACTACTGGGGCCAAGGAACCTCAGTCACCG
TCTCCTCAACCACGACGCCAGCGCCGCGACCACCAACACCGGCGCCCACCATCGCGTCGCA
GCCCCTGTCCCTGCGCCCAGAGGCGTGCCGGCCAGCGGCGGGGGGCGCAGTGCACACGAG
GGGGCTGGACTTCGCCTGTGATATCTACATCTGGGCGCCCTTGGCCGGGACTTGTGGGGTCC
TTCTCCTGTCACTGGTTATCACCCTTTACTGCAAACGGGGCAGAAAGAAACTCCTGTATATATTC
AAACAACCATTTATGAGACCAGTACAAACTACTCAAGAGGAAGATGGCTGTAGCTGCCGATTTC
CAGAAGAAGAAGAAGGAGGATGTGAACTGAGAGTGAAGTTCAGCAGGAGCGCAGACGCCCC
CGCGTACAAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGAGAAGGAA
GAACCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCGC.

Herein, the nucleic acid molecule with the nucleotide sequence shown in SEQ ID NO: 13 encodes a chimeric antigen receptor expressed by Car-pCDHF32 (the molecular structure is shown in FIG. 1), the nucleic acid molecule with the nucleotide sequence shown in SEQ ID NO: 14 encodes a chimeric antigen receptor expressed by Car-pCDHF34 (the molecular structure is shown in FIG. 2), the nucleic acid molecule with the nucleotide sequence shown in SEQ ID NO: 15 encodes a chimeric antigen receptor expressed by Car-pCDHF31 (the molecular structure is shown in FIG. 15), the nucleic acid molecule with the nucleotide sequence shown in SEQ ID NO: 16 encodes a chimeric antigen receptor expressed by Car-pCDHF58, and the nucleic acid molecule with the nucleotide sequence shown in SEQ ID NO: 17 encodes a chimeric antigen receptor expressed by Car-pCDH F59.

In a third aspect of the present disclosure, the present disclosure provides a lentivirus. According to an embodiment of the present disclosure, the lentivirus carries a nucleic acid molecule with a nucleotide sequence shown in SEQ ID NO: 18˜22. After the lentivirus according to the embodiment of the present disclosure is introduced into a receptor cell-T lymphocyte, the T lymphocyte obtained may specifically kill the single/double positive tumor cells of the first antigen and the second antigen, and has the longer and more powerful effect of killing and eliminating the tumor cells in vivo compared with the existing technology.

(SEQ ID NO: 18)
CGTGAGGCTCCGGTGCCCGTCAGTGGGCAGAGCGCACATCGCCCACAGTCCCCGAGAA
GTTGGGGGGAGGGGTCGGCAATTGACCCGGTGCCTAGAGAAGGTGGCGCGGGGTAAACTGG
GAAAGTGATGTCGTGTACTGGCTCCGCCTTTTTCCCGAGGGTGGGGGAGAACCGTATATAAGT
GCAGTAGTCGCCGTGAACGTTCTTTTTCGCAACGGGTTTGCCGCCAGAACACAGGTAAGTGC
CGTGTGTGGTTCCCGCGGGCCTGGCCTCTTTACGGGTTATGGCCCTTGCGTGCCTTGAATTAC
TTCCACCTGGCTGCAGTACGTGATTCTTGATCCCGAGCTTCGGGTTGGAAGTGGGTGGGAGA
GTTCGAGGCCTTGCGCTTAAGGAGCCCCTTCGCCTCGTGCTTGAGTTGAGGCCTGGCCTGG
GCGCTGGGGCCGCCGCGTGCGAATCTGGTGGCACCTTCGCGCCTGTCTCGCTGCTTTCGATA
AGTCTCTAGCCATTTAAAATTTTTGATGACCTGCTGCGACGCTTTTTTTCTGGCAAGATAGTCTT
GTAAATGCGGGCCAAGATCTGCACACTGGTATTTCGGTTTTTGGGGCCGCGGGCGGCGACGG
GGCCCGTGCGTCCCAGCGCACATGTTCGGCGAGGCGGGGCCTGCGAGCGCGGCCACCGAG
AATCGGACGGGGGTAGTCTCAAGCTCGCCGGCCTGCTCTGGTGCCTGGCCTCGCGCCGCCG
TGTATCGCCCCGCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGGCGCTCGGGAGA
GCGGGCGGGTGAGTCACCCACACAAAGGAAAAGGGCCTTTCCGTCCTCAGCCGTCGCTTCAT
GTGACTCCACTGAGTACCGGGCGCCGTCCAGGCACCTCGATTAGTTCTCGAGCTTTTGGAGT
ACGTCGTCTTTAGGTTGGGGGGAGGGGTTTTATGCGATGGAGTTTCCCCACACTGAGTGGGT
GGAGACTGAAGTTAGGCCAGCTTGGCACTTGATGTAATTCTCCTTGGAATTTGCCCTTTTTGAG
TTTGGATCTTGGTTCATTCTCAAGCCTCAGACAGTGGTTCAAAGTTTTTTTCTTCCATTTCAGGT
GTCGTGAGGGATCCCGCCACCATGGCCTTACCAGTGACCGCCTTGCTCCTGCCGCTGGCCTT
GCTGCTCCACGCCGCCAGGCCGCAGGTGCAGCTGCAGCAGAGCGGACCAGGACTGGTGAA
GCCATCTCAGACCCTGAGCCTGACATGCGCCATCTCTGGCGATAGCGTGAGCTCCAACTCTG
CCGCCTGGAATTGGATCAGACAGTCTCCAAGCAGAGGACTGGAGTGGCTGGGAAGGACCTAC
TATAGATCCAAGTGGTACAACGATTATGCCGTGTCCGTGAAGTCTCGGATCACCATCAACCCTG
ACACATCCAAGAATCAGTTCTCTCTGCAGCTGAATAGCGTGACCCCAGAGGACACAGCCGTGT
ACTATTGCGCCCGGGAGGTGACCGGCGACCTGGAGGATGCCTTTGACATCTGGGGCCAGGG
CACCATGGTGACAGTGGGAGGAGGAGGCTCCGGCGGAGGAGGCTCTGGAGGAGGAGGCAG
CGACATCCAGATGACCCAGTCCCCATCTAGCCTGTCCGCCTCTGTGGGCGACAGGGTGACCA
TCACATGTCGCGCCTCCCAGACAATCTGGTCTTACCTGAACTGGTATCAGCAGAGACCCGGCA
AGGCCCCTAATCTGCTGATCTACGCAGCCTCCTCTCTGCAGAGCGGAGTGCCAAGCAGGTTC
TCCGGCCGCGGCTCCGGCACCGACTTCACCCTGACAATCAGCTCCCTGCAGGCCGAGGACT
TCGCCACATACTATTGTCAGCAGAGCTATTCCATCCCTCAGACCTTTGGCCAGGGCACAAAGC
TGGAGATCAAGGGAGGCGGTGGGTCGGACATCCAGATGACACAGACTACATCCTCCCTGTCT
GCCTCTCTGGGAGACAGAGTCACCATCAGTTGCAGGGCAAGTCAGGACATTAGTAAATATTTAA
ATTGGTATCAGCAGAAACCAGATGGAACTGTTAAACTCCTGATCTACCATACATCAAGATTACAC
TCAGGAGTCCCATCAAGGTTCAGTGGCAGTGGGTCTGGAACAGATTATTCTCTCACCATTAGC
AACCTGGAGCAAGAAGATATTGCCACTTACTTTTGCCAACAGGGTAATACGCTTCCGTACACGT
TCGGAGGGGGGACCAAGCTGGAGATCACAGGTGGCGGTGGCTCGGGCGGTGGTGGGTCGG
GTGGCGGCGGATCTGAGGTGAAACTGCAGGAGTCAGGACCTGGCCTGGTGGCGCCCTCACA
GAGCCTGTCCGTCACATGCACTGTCTCAGGGGTCTCATTACCCGACTATGGTGTAAGCTGGAT
TCGCCAGCCTCCACGAAAGGGTCTGGAGTGGCTGGGAGTAATATGGGGTAGTGAAACCACAT
ACTATAATTCAGCTCTCAAATCCAGACTGACCATCATCAAGGACAACTCCAAGAGCCAAGTTTT
CTTAAAAATGAACAGTCTGCAAACTGATGACACAGCCATTTACTACTGTGCCAAACATTATTACT
ACGGTGGTAGCTATGCTATGGACTACTGGGGCCAAGGAACCTCAGTCACCGTCTCCTCAACCA
CGACGCCAGCGCCGCGACCACCAACACCGGCGCCCACCATCGCGTCGCAGCCCCTGTCCCT
GCGCCCAGAGGCGTGCCGGCCAGCGGCGGGGGGCGCAGTGCACACGAGGGGGCTGGACT
TCGCCTGTGATATCTACATCTGGGCGCCCTTGGCCGGGACTTGTGGGGTCCTTCTCCTGTCAC
TGGTTATCACCCTTTACTGCAAACGGGGCAGAAAGAAACTCCTGTATATATTCAAACAACCATTT
ATGAGACCAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTACAAGC
AGGGCCAGAACCAGCTCTATAACGAGCTCAATCTAGGACGAAGAGAGGAGTACGATGTTTTGG
ACAAGAGACGTGGCCGGGACCCTGAGATGGGGGGAAAGCCGAGAAGGAAGAACCCTCAGGA
AGGCCTGTACAATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTACAGCCACC
AAGGACACCTACGACGCCCTTCACATGCAGGCCCTGCCCCCTCGC.
(SEQ ID NO: 19)
CGTGAGGCTCCGGTGCCCGTCAGTGGGCAGAGCGCACATCGCCCACAGTCCCCGAGAA
GTTGGGGGGAGGGGTCGGCAATTGACCCGGTGCCTAGAGAAGGTGGCGCGGGGTAAACTGG
GAAAGTGATGTCGTGTACTGGCTCCGCCTTTTTCCCGAGGGTGGGGGAGAACCGTATATAAGT
GCAGTAGTCGCCGTGAACGTTCTTTTTCGCAACGGGTTTGCCGCCAGAACACAGGTAAGTGC
CGTGTGTGGTTCCCGCGGGCCTGGCCTCTTTACGGGTTATGGCCCTTGCGTGCCTTGAATTAC
TTCCACCTGGCTGCAGTACGTGATTCTTGATCCCGAGCTTCGGGTTGGAAGTGGGTGGGAGA
GTTCGAGGCCTTGCGCTTAAGGAGCCCCTTCGCCTCGTGCTTGAGTTGAGGCCTGGCCTGG
GCGCTGGGGCCGCCGCGTGCGAATCTGGTGGCACCTTCGCGCCTGTCTCGCTGCTTTCGATA
AGTCTCTAGCCATTTAAAATTTTTGATGACCTGCTGCGACGCTTTTTTTCTGGCAAGATAGTCTT
GTAAATGCGGGCCAAGATCTGCACACTGGTATTTCGGTTTTTGGGGCCGCGGGCGGCGACGG
GGCCCGTGCGTCCCAGCGCACATGTTCGGCGAGGCGGGGCCTGCGAGCGCGGCCACCGAG
AATCGGACGGGGGTAGTCTCAAGCTCGCCGGCCTGCTCTGGTGCCTGGCCTCGCGCCGCCG
TGTATCGCCCCGCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGGCGCTCGGGAGA
GCGGGCGGGTGAGTCACCCACACAAAGGAAAAGGGCCTTTCCGTCCTCAGCCGTCGCTTCAT
GTGACTCCACTGAGTACCGGGCGCCGTCCAGGCACCTCGATTAGTTCTCGAGCTTTTGGAGT
ACGTCGTCTTTAGGTTGGGGGGAGGGGTTTTATGCGATGGAGTTTCCCCACACTGAGTGGGT
GGAGACTGAAGTTAGGCCAGCTTGGCACTTGATGTAATTCTCCTTGGAATTTGCCCTTTTTGAG
TTTGGATCTTGGTTCATTCTCAAGCCTCAGACAGTGGTTCAAAGTTTTTTTCTTCCATTTCAGGT
GTCGTGAGGGATCCCGCCACCATGGCCTTACCAGTGACCGCCTTGCTCCTGCCGCTGGCCTT
GCTGCTCCACGCCGCCAGGCCGGACATCCAGATGACACAGACTACATCCTCCCTGTCTGCCT
CTCTGGGAGACAGAGTCACCATCAGTTGCAGGGCAAGTCAGGACATTAGTAAATATTTAAATTG
GTATCAGCAGAAACCAGATGGAACTGTTAAACTCCTGATCTACCATACATCAAGATTACACTCAG
GAGTCCCATCAAGGTTCAGTGGCAGTGGGTCTGGAACAGATTATTCTCTCACCATTAGCAACC
TGGAGCAAGAAGATATTGCCACTTACTTTTGCCAACAGGGTAATACGCTTCCGTACACGTTCGG
AGGGGGGACCAAGCTGGAGATCACAGGTGGCGGTGGCTCGGGCGGTGGTGGGTCGGGTGG
CGGCGGATCTGAGGTGAAACTGCAGGAGTCAGGACCTGGCCTGGTGGCGCCCTCACAGAGC
CTGTCCGTCACATGCACTGTCTCAGGGGTCTCATTACCCGACTATGGTGTAAGCTGGATTCGC
CAGCCTCCACGAAAGGGTCTGGAGTGGCTGGGAGTAATATGGGGTAGTGAAACCACATACTAT
AATTCAGCTCTCAAATCCAGACTGACCATCATCAAGGACAACTCCAAGAGCCAAGTTTTCTTAA
AAATGAACAGTCTGCAAACTGATGACACAGCCATTTACTACTGTGCCAAACATTATTACTACGGT
GGTAGCTATGCTATGGACTACTGGGGCCAAGGAACCTCAGTCACCGTCTCCTCAGGAGGCGG
TGGGTCGCAGGTGCAGCTGCAGCAGAGCGGACCAGGACTGGTGAAGCCATCTCAGACCCTG
AGCCTGACATGCGCCATCTCTGGCGATAGCGTGAGCTCCAACTCTGCCGCCTGGAATTGGATC
AGACAGTCTCCAAGCAGAGGACTGGAGTGGCTGGGAAGGACCTACTATAGATCCAAGTGGTA
CAACGATTATGCCGTGTCCGTGAAGTCTCGGATCACCATCAACCCTGACACATCCAAGAATCA
GTTCTCTCTGCAGCTGAATAGCGTGACCCCAGAGGACACAGCCGTGTACTATTGCGCCCGGG
AGGTGACCGGCGACCTGGAGGATGCCTTTGACATCTGGGGCCAGGGCACCATGGTGACAGT
GGGAGGAGGAGGCTCCGGCGGAGGAGGCTCTGGAGGAGGAGGCAGCGACATCCAGATGAC
CCAGTCCCCATCTAGCCTGTCCGCCTCTGTGGGCGACAGGGTGACCATCACATGTCGCGCCT
CCCAGACAATCTGGTCTTACCTGAACTGGTATCAGCAGAGACCCGGCAAGGCCCCTAATCTGC
TGATCTACGCAGCCTCCTCTCTGCAGAGCGGAGTGCCAAGCAGGTTCTCCGGCCGCGGCTCC
GGCACCGACTTCACCCTGACAATCAGCTCCCTGCAGGCCGAGGACTTCGCCACATACTATTGT
CAGCAGAGCTATTCCATCCCTCAGACCTTTGGCCAGGGCACAAAGCTGGAGATCAAGACCAC
GACGCCAGCGCCGCGACCACCAACACCGGCGCCCACCATCGCGTCGCAGCCCCTGTCCCTG
CGCCCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGTCACT
GGTTATCACCCTTTACTGCAAACGGGGCAGAAAGAAACTCCTGTATATATTCAAACAACCATTTA
TGAGACCAGTACAAACTACTCAAGAGGAAGATGGCTGTAGCTGCCGATTTCCAGAAGAAGAAG
AAGGAGGATGTGAACTGAGAGTGAAGTTCAGCAGGAGCGCAGACGCCCCCGCGTACAAGCA
GGGCCAGAACCAGCTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGAACCCTCAGGAA
GGCCTGTACAATGAACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAGTACAGCCACCA
AGGACACCTACGACGCCCTTCACATGCAGGCCCTGCCCCCTCGC.
(SEQ ID NO: 20)
CGTGAGGCTCCGGTGCCCGTCAGTGGGCAGAGCGCACATCGCCCACAGTCCCCGAGAA
GTTGGGGGGAGGGGTCGGCAATTGACCCGGTGCCTAGAGAAGGTGGCGCGGGGTAAACTGG
GAAAGTGATGTCGTGTACTGGCTCCGCCTTTTTCCCGAGGGTGGGGGAGAACCGTATATAAGT
GCAGTAGTCGCCGTGAACGTTCTTTTTCGCAACGGGTTTGCCGCCAGAACACAGGTAAGTGC
CGTGTGTGGTTCCCGCGGGCCTGGCCTCTTTACGGGTTATGGCCCTTGCGTGCCTTGAATTAC
TTCCACCTGGCTGCAGTACGTGATTCTTGATCCCGAGCTTCGGGTTGGAAGTGGGTGGGAGA
GTTCGAGGCCTTGCGCTTAAGGAGCCCCTTCGCCTCGTGCTTGAGTTGAGGCCTGGCCTGG
GCGCTGGGGCCGCCGCGTGCGAATCTGGTGGCACCTTCGCGCCTGTCTCGCTGCTTTCGATA
AGTCTCTAGCCATTTAAAATTTTTGATGACCTGCTGCGACGCTTTTTTTCTGGCAAGATAGTCTT
GTAAATGCGGGCCAAGATCTGCACACTGGTATTTCGGTTTTTGGGGCCGCGGGCGGCGACGG
GGCCCGTGCGTCCCAGCGCACATGTTCGGCGAGGCGGGGCCTGCGAGCGCGGCCACCGAG
AATCGGACGGGGGTAGTCTCAAGCTCGCCGGCCTGCTCTGGTGCCTGGCCTCGCGCCGCCG
TGTATCGCCCCGCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGGCGCTCGGGAGA
GCGGGCGGGTGAGTCACCCACACAAAGGAAAAGGGCCTTTCCGTCCTCAGCCGTCGCTTCAT
GTGACTCCACTGAGTACCGGGCGCCGTCCAGGCACCTCGATTAGTTCTCGAGCTTTTGGAGT
ACGTCGTCTTTAGGTTGGGGGGAGGGGTTTTATGCGATGGAGTTTCCCCACACTGAGTGGGT
GGAGACTGAAGTTAGGCCAGCTTGGCACTTGATGTAATTCTCCTTGGAATTTGCCCTTTTTGAG
TTTGGATCTTGGTTCATTCTCAAGCCTCAGACAGTGGTTCAAAGTTTTTTTCTTCCATTTCAGGT
GTCGTGAGGGATCCCGCCACCATGGCCTTACCAGTGACCGCCTTGCTCCTGCCGCTGGCCTT
GCTGCTCCACGCCGCCAGGCCGCAGATCCAGCTGGTGCAGTCTGGCCCAGAGCTGAAGAAG
CCCGGCGAGACCGTGAAGATCAGCTGCAAGGCCTCCGGCTACACCTTCACAGACTATAGCAT
CAACTGGGTGAAGAGGGCCCCTGGCAAGGGCCTGAAGTGGATGGGCTGGATCAATACCGAG
ACACGCGAGCCAGCCTACGCCTATGACTTCCGGGGCAGATTCGCCTTTTCCCTGGAGACCTC
TGCCAGCACAGCCTACCTGCAGATCAACAATCTGAAGTACGAGGATACCGCCACATATTTTTGC
GCCCTGGACTACAGCTATGCCATGGATTATTGGGGCCAGGGCACCTCCGTGACAGTGAGCTC
CGGAGGAGGAGGCTCCGGCGGCGGAGGCTCTGGCGGCGGCGGCAGCGACATCGTGCTGAC
CCAGTCCCCAGCCTCTCTGGCCATGTCCCTGGGCAAGCGGGCCACAATCTCTTGTAGAGCCT
CCGAGTCTGTGAGCGTGATCGGCGCCCACCTGATCCACTGGTACCAGCAGAAGCCTGGCCA
GCCCCCTAAGCTGCTGATCTATCTGGCCAGCAACCTGGAGACCGGAGTGCCAGCACGGTTCT
CCGGCTCTGGCAGCGGCACAGACTTTACCCTGACAATCGATCCTGTGGAGGAGGACGATGTG
GCCATCTACTCTTGTCTGCAGAGCAGGATCTTCCCACGCACCTTTGGCGGCGGCACAAAGCT
GGAGATCAAGGGAGGCGGTGGGTCGGACATCCAGATGACACAGACTACATCCTCCCTGTCTG
CCTCTCTGGGAGACAGAGTCACCATCAGTTGCAGGGCAAGTCAGGACATTAGTAAATATTTAAA
TTGGTATCAGCAGAAACCAGATGGAACTGTTAAACTCCTGATCTACCATACATCAAGATTACACT
CAGGAGTCCCATCAAGGTTCAGTGGCAGTGGGTCTGGAACAGATTATTCTCTCACCATTAGCA
ACCTGGAGCAAGAAGATATTGCCACTTACTTTTGCCAACAGGGTAATACGCTTCCGTACACGTT
CGGAGGGGGGACCAAGCTGGAGATCACAGGTGGCGGTGGCTCGGGCGGTGGTGGGTCGGG
TGGCGGCGGATCTGAGGTGAAACTGCAGGAGTCAGGACCTGGCCTGGTGGCGCCCTCACAG
AGCCTGTCCGTCACATGCACTGTCTCAGGGGTCTCATTACCCGACTATGGTGTAAGCTGGATT
CGCCAGCCTCCACGAAAGGGTCTGGAGTGGCTGGGAGTAATATGGGGTAGTGAAACCACATA
CTATAATTCAGCTCTCAAATCCAGACTGACCATCATCAAGGACAACTCCAAGAGCCAAGTTTTC
TTAAAAATGAACAGTCTGCAAACTGATGACACAGCCATTTACTACTGTGCCAAACATTATTACTA
CGGTGGTAGCTATGCTATGGACTACTGGGGCCAAGGAACCTCAGTCACCGTCTCCTCAACCAC
GACGCCAGCGCCGCGACCACCAACACCGGCGCCCACCATCGCGTCGCAGCCCCTGTCCCTG
CGCCCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGTCACT
GGTTATCACCCTTTACTGCAAACGGGGCAGAAAGAAACTCCTGTATATATTCAAACAACCATTTA
TGAGACCAGTACAAACTACTCAAGAGGAAGATGGCTGTAGCTGCCGATTTCCAGAAGAAGAAG
AAGGAGGATGTGAACTGAGAGTGAAGTTCAGCAGGAGCGCAGACGCCCCCGCGTACAAGCA
GGGCCAGAACCAGCTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGAACCCTCAGGAA
GGCCTGTACAATGAACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAGTACAGCCACCA
AGGACACCTACGACGCCCTTCACATGCAGGCCCTGCCCCCTCGCTAATGAGGCCGGCC.
(SEQ ID NO: 21)
GCCACCATGGCCTTACCAGTGACCGCCTTGCTCCTGCCGCTGGCCTTGCTGCTCCACGC
CGCCAGGCCGCAGGTGCAGCTGCAGCAGAGCGGACCAGGACTGGTGAAGCCATCTCAGACC
CTGAGCCTGACATGCGCCATCTCTGGCGATAGCGTGAGCTCCAACTCTGCCGCCTGGAATTG
GATCAGACAGTCTCCAAGCAGAGGACTGGAGTGGCTGGGAAGGACCTACTATAGATCCAAGT
GGTACAACGATTATGCCGTGTCCGTGAAGTCTCGGATCACCATCAACCCTGACACATCCAAGA
ATCAGTTCTCTCTGCAGCTGAATAGCGTGACCCCAGAGGACACAGCCGTGTACTATTGCGCCC
GGGAGGTGACCGGCGACCTGGAGGATGCCTTTGACATCTGGGGCCAGGGCACCATGGTGAC
AGTGGGCGGAGGAGGCTCTGGCGGAGGAGGCTCTGGAGGAGGAGGCAGCGGCGGAGGAG
GCTCTGGAGGAGGAGGCAGCGACATCCAGATGACCCAGTCCCCATCTAGCCTGTCCGCCTCT
GTGGGCGACAGGGTGACCATCACATGTCGCGCCTCCCAGACAATCTGGTCTTACCTGAACTG
GTATCAGCAGAGACCCGGCAAGGCCCCTAATCTGCTGATCTACGCAGCCTCCTCTCTGCAGAG
CGGAGTGCCAAGCAGGTTCTCCGGCCGCGGCTCCGGCACCGACTTCACCCTGACAATCAGC
TCCCTGCAGGCCGAGGACTTCGCCACATACTATTGTCAGCAGAGCTATTCCATCCCTCAGACC
TTTGGCCAGGGCACAAAGCTGGAGATCAAGGGAGGCGGTGGGTCGGACATCCAGATGACAC
AGACTACATCCTCCCTGTCTGCCTCTCTGGGAGACAGAGTCACCATCAGTTGCAGGGCAAGTC
AGGACATTAGTAAATATTTAAATTGGTATCAGCAGAAACCAGATGGAACTGTTAAACTCCTGATCT
ACCATACATCAAGATTACACTCAGGAGTCCCATCAAGGTTCAGTGGCAGTGGGTCTGGAACAG
ATTATTCTCTCACCATTAGCAACCTGGAGCAAGAAGATATTGCCACTTACTTTTGCCAACAGGGT
AATACGCTTCCGTACACGTTCGGAGGGGGGACCAAGCTGGAGATCACAGGCGGTGGTGGGT
CGGGCGGTGGTGGGTCGGGCGGAGGAGGCTCTGGAGGAGGAGGCAGCGGTGGCGGCGGA
TCTGAGGTGAAACTGCAGGAGTCAGGACCTGGCCTGGTGGCGCCCTCACAGAGCCTGTCCG
TCACATGCACTGTCTCAGGGGTCTCATTACCCGACTATGGTGTAAGCTGGATTCGCCAGCCTC
CACGAAAGGGTCTGGAGTGGCTGGGAGTAATATGGGGTAGTGAAACCACATACTATAATTCAG
CTCTCAAATCCAGACTGACCATCATCAAGGACAACTCCAAGAGCCAAGTTTTCTTAAAAATGAA
CAGTCTGCAAACTGATGACACAGCCATTTACTACTGTGCCAAACATTATTACTACGGTGGTAGCT
ATGCTATGGACTACTGGGGCCAAGGAACCTCAGTCACCGTCTCCTCAACCACGACGCCAGCG
CCGCGACCACCAACACCGGCGCCCACCATCGCGTCGCAGCCCCTGTCCCTGCGCCCAGAGG
CGTGCCGGCCAGCGGCGGGGGGCGCAGTGCACACGAGGGGGCTGGACTTCGCCTGTGATA
TCTACATCTGGGCGCCCTTGGCCGGGACTTGTGGGGTCCTTCTCCTGTCACTGGTTATCACCC
TTTACTGCAAACGGGGCAGAAAGAAACTCCTGTATATATTCAAACAACCATTTATGAGACCAGTA
CAAACTACTCAAGAGGAAGATGGCTGTAGCTGCCGATTTCCAGAAGAAGAAGAAGGAGGATGT
GAACTGAGAGTGAAGTTCAGCAGGAGCGCAGACGCCCCCGCGTACAAGCAGGGCCAGAACC
AGCTCTATAACGAGCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGGACACCTA
CGACGCCCTTCACATGCAGGCCCTGCCCCCTCGCTAA.
(SEQ ID NO: 22)
GCCACCATGGCCTTACCAGTGACCGCCTTGCTCCTGCCGCTGGCCTTGCTGCTCCACGC
CGCCAGGCCGCAGGTGCAGCTGCAGCAGAGCGGACCAGGACTGGTGAAGCCATCTCAGACC
CTGAGCCTGACATGCGCCATCTCTGGCGATAGCGTGAGCTCCAACTCTGCCGCCTGGAATTG
GATCAGACAGTCTCCAAGCAGAGGACTGGAGTGGCTGGGAAGGACCTACTATAGATCCAAGT
GGTACAACGATTATGCCGTGTCCGTGAAGTCTCGGATCACCATCAACCCTGACACATCCAAGA
ATCAGTTCTCTCTGCAGCTGAATAGCGTGACCCCAGAGGACACAGCCGTGTACTATTGCGCCC
GGGAGGTGACCGGCGACCTGGAGGATGCCTTTGACATCTGGGGCCAGGGCACCATGGTGAC
AGTGGGCGGAGGAGGCTCTGGCGGTGGTGGGTCGGGCGGAGGAGGCTCTGGAGGAGGAG
GCAGCGGCGGAGGAGGCTCTGGAGGAGGAGGCAGCGACATCCAGATGACCCAGTCCCCATC
TAGCCTGTCCGCCTCTGTGGGCGACAGGGTGACCATCACATGTCGCGCCTCCCAGACAATCT
GGTCTTACCTGAACTGGTATCAGCAGAGACCCGGCAAGGCCCCTAATCTGCTGATCTACGCAG
CCTCCTCTCTGCAGAGCGGAGTGCCAAGCAGGTTCTCCGGCCGCGGCTCCGGCACCGACTT
CACCCTGACAATCAGCTCCCTGCAGGCCGAGGACTTCGCCACATACTATTGTCAGCAGAGCTA
TTCCATCCCTCAGACCTTTGGCCAGGGCACAAAGCTGGAGATCAAGGGAGGCGGTGGGTCG
GACATCCAGATGACACAGACTACATCCTCCCTGTCTGCCTCTCTGGGAGACAGAGTCACCATC
AGTTGCAGGGCAAGTCAGGACATTAGTAAATATTTAAATTGGTATCAGCAGAAACCAGATGGAA
CTGTTAAACTCCTGATCTACCATACATCAAGATTACACTCAGGAGTCCCATCAAGGTTCAGTGG
CAGTGGGTCTGGAACAGATTATTCTCTCACCATTAGCAACCTGGAGCAAGAAGATATTGCCACT
TACTTTTGCCAACAGGGTAATACGCTTCCGTACACGTTCGGAGGGGGGACCAAGCTGGAGATC
ACAGGCGGTGGTGGGTCGGGCGGTGGTGGGTCGGGCGGTGGTGGGTCGGGCGGAGGAGG
CTCTGGAGGAGGAGGCAGCGGTGGCGGCGGATCTGAGGTGAAACTGCAGGAGTCAGGACCT
GGCCTGGTGGCGCCCTCACAGAGCCTGTCCGTCACATGCACTGTCTCAGGGGTCTCATTACC
CGACTATGGTGTAAGCTGGATTCGCCAGCCTCCACGAAAGGGTCTGGAGTGGCTGGGAGTAA
TATGGGGTAGTGAAACCACATACTATAATTCAGCTCTCAAATCCAGACTGACCATCATCAAGGAC
AACTCCAAGAGCCAAGTTTTCTTAAAAATGAACAGTCTGCAAACTGATGACACAGCCATTTACT
ACTGTGCCAAACATTATTACTACGGTGGTAGCTATGCTATGGACTACTGGGGCCAAGGAACCTC
AGTCACCGTCTCCTCAACCACGACGCCAGCGCCGCGACCACCAACACCGGCGCCCACCATC
GCGTCGCAGCCCCTGTCCCTGCGCCCAGAGGCGTGCCGGCCAGCGGCGGGGGGCGCAGT
GCACACGAGGGGGCTGGACTTCGCCTGTGATATCTACATCTGGGCGCCCTTGGCCGGGACTT
GTGGGGTCCTTCTCCTGTCACTGGTTATCACCCTTTACTGCAAACGGGGCAGAAAGAAACTCC
TGTATATATTCAAACAACCATTTATGAGACCAGTACAAACTACTCAAGAGGAAGATGGCTGTAGC
TGCCGATTTCCAGAAGAAGAAGAAGGAGGATGTGAACTGAGAGTGAAGTTCAGCAGGAGCGC
AGACGCCCCCGCGTACAAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCTAA.

Herein, the nucleic acid molecule with the nucleotide sequence shown in SEQ ID NO: 18 expresses a Car-pCDHF32 (the molecular structure is shown in FIG. 1) chimeric antigen receptor, the nucleic acid molecule with the nucleotide sequence shown in SEQ ID NO: 19 expresses a Car-pCDHF34 (the molecular structure is shown in FIG. 2) chimeric antigen receptor, the nucleic acid molecule with the nucleotide sequence shown in SEQ ID NO: 20 expresses a Car-pCDHF31 (the molecular structure is shown in FIG. 15) chimeric antigen receptor, the nucleic acid molecule with the nucleotide sequence shown in SEQ ID NO: 21 expresses a Car-pCDHF58 chimeric antigen receptor, and the nucleic acid molecule with the nucleotide sequence shown in SEQ ID NO: 22 expresses a Car-pCDHF59 chimeric antigen receptor.

In a fourth aspect of the present disclosure, the present disclosure provides a transgenic lymphocyte. According to an embodiment of the present disclosure, the lymphocyte expresses a chimeric antigen receptor, and the chimeric antigen receptor includes: an extracellular region, herein the extracellular region includes a first single-chain antibody, a second single-chain antibody, and a first linker peptide, the first single-chain antibody specifically recognizes a first antigen, the second single-chain antibody specifically recognizes a second antigen, the first linker peptide is arranged between the first single-chain antibody and the second single-chain antibody, the first single-chain antibody includes a first heavy chain variable region, a first light chain variable region and a second linker peptide, the second linker peptide is arranged between the first heavy chain variable region and the first light chain variable region, the second single-chain antibody includes a second heavy chain variable region, a second light chain variable region and a third linker peptide, the third linker peptide is arranged between the second heavy chain variable region and the second light chain variable region, the first linker peptide has a repeated amino acid sequence of GGGGS, and the second linker peptide and the third linker peptide independently have 2˜6 repeated amino acid sequences of GGGGS respectively; a transmembrane region, herein the transmembrane region is connected with the extracellular region, and is embedded into a cell membrane of the lymphocyte; and an intracellular region, herein the intracellular region is connected with the transmembrane region, and the intracellular region includes an intracellular segment of an immune co-stimulator molecule. The transgenic lymphocyte according to the embodiment of the present disclosure may specifically kill the single/double positive tumor cells of the first antigen and the second antigen, and has the longer and more powerful effect of killing and eliminating the tumor cells in vivo compared with the existing technology.

According to an embodiment of the present disclosure, the above transgenic lymphocyte may further include the following additional technical features.

According to an embodiment of the disclosure, the first single-chain antibody is a CD19 single-chain antibody, the second single-chain antibody is a CD22 single-chain antibody, the first antigen is CD19, the second antigen is CD22, the first heavy chain variable region is a CD19 heavy chain variable region, the first light chain variable region is a CD19 light chain variable region, the second heavy chain variable region is a CD22 heavy chain variable region, and the second light chain variable region is a CD22 light chain variable region.

According to an embodiment of the present disclosure, the first single-chain antibody is a CD19 single-chain antibody, the second single-chain antibody is a BCMA single-chain antibody, the first antigen is CD19, the second antigen is BCMA, the first heavy chain variable region is a CD19 heavy chain variable region, the first light chain variable region is a CD19 light chain variable region, the second heavy chain variable region is a BCMA heavy chain variable region, and the second light chain variable region is a BCMA light chain variable region.

According to an embodiment of the present disclosure, the intracellular segment of the immune co-stimulatory molecule is independently selected from at least one of 4-1 BB, OX-40, CD40L, CD27, CD30, CD28 and their derivatives.

According to an embodiment of the present disclosure, the intracellular segment of the immune co-stimulatory molecule is an intracellular segment of 4-1 BB and CD3.

According to an embodiment of the present disclosure, the lymphocyte is a CD3⁺ T lymphocyte.

According to an embodiment of the present disclosure, the lymphocyte is a CD8⁺ T lymphocyte.

According to an embodiment of the present disclosure, the lymphocyte is a natural killer cell.

According to an embodiment of the present disclosure, the lymphocyte is a natural killer T cell.

According to an embodiment of the present disclosure, the N terminal of the first linker peptide is connected with the C terminal of the second single-chain antibody, the C terminal of the first linker peptide is connected with the N terminal of the first single-chain antibody, and the C terminal of the first single-chain antibody is connected with the N terminal of the CD8 transmembrane region. It is found by the inventor that in the above connecting order of the second single-chain antibody, the first linker peptide and the first single-chain antibody, the secretion of cytokines, such as IL-2 and IFN-γ, is lower, and in vivo treatment of the transgenic lymphocyte according to the embodiment of the present disclosure is safer.

According to an embodiment of the present disclosure, the N terminal of the first linker peptide is connected with the C terminal of the CD22 single-chain antibody, the C terminal of the first linker peptide is connected with the N terminal of the CD19 single-chain antibody, and the C terminal of the CD19 single-chain antibody is connected with the N terminal of the transmembrane region. The above transgenic lymphocyte according to the embodiment of the present disclosure may specifically kill the single/double positive tumor cells of CD19 and CD22, and the cytokine secretion in vivo is lower. Compared with the existing technology, it has the longer and more powerful effect of killing and eliminating blood tumor cells in vivo.

According to an embodiment of the present disclosure, the N terminal of the first linker peptide is connected with the C terminal of the CD19 single-chain antibody, the C terminal of the first linker peptide is connected with the N terminal of the CD22 single-chain antibody, and the C terminal of the CD22 single-chain antibody is connected with the N terminal of the transmembrane region. The above transgenic according to the embodiment of the present disclosure may specifically kill the single/double positive tumor cells of CD19 and CD22, and has the longer and more powerful effect of killing and eliminating the blood tumor cells in vivo compared with the existing technology.

According to an embodiment of the present disclosure, the N terminal of the third linker peptide is connected with the C terminal of the CD22 heavy chain variable region, the C terminal of the third linker peptide is connected with the N terminal of the CD22 light chain variable region, the C terminal of the CD22 light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the transmembrane region. The above transgenic lymphocyte according to the embodiment of the present disclosure may specifically kill the single/double positive tumor cells of CD19 and CD22, and the cytokine secretion in vivo is lower. Compared with the existing technology, it has the longer and more powerful effect of killing and eliminating the blood tumor cells in vivo.

According to an embodiment of the present disclosure, the N terminal of the second linker peptide is connected with the C terminal of the CD19 light chain variable region, the C terminal of the second linker peptide is connected with the N terminal of the CD19 heavy chain variable region, the C terminal of the CD19 heavy chain variable region is connected with the N terminal of the first linker peptide, the C terminal of the first linker peptide is connected with the N terminal of the CD22 heavy chain variable region, the N terminal of the third linker peptide is connected with the C terminal of the CD22 heavy chain variable region, and the C terminal of the third linker peptide is connected with the N terminal of the CD22 light chain variable region, and the C terminal of the CD22 light chain variable region is connected with the N terminal of the transmembrane region. The above transgenic lymphocyte according to the embodiment of the present disclosure may specifically kill the single/double positive tumor cells of CD19 and CD22, and has the longer and more powerful effect of killing and eliminating the blood tumor cells in vivo compared with the existing technology.

According to an embodiment of the present disclosure, the N terminal of the third linker peptide is connected with the C terminal of the BCMA heavy chain variable region, the C terminal of the third linker peptide is connected with the N terminal of the BCMA light chain variable region, the C terminal of the BCMA light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region. The above transgenic lymphocyte according to the embodiment of the present disclosure may specifically kill the single/double positive tumor cells of BCMA and CD19, and has the longer and more powerful effect of killing and eliminating the tumor cells in vivo compared with the existing technology.

According to an embodiment of the present disclosure, the second linker peptide and the third linker peptide independently have 3˜5 repeated amino acid sequences of GGGGS respectively.

According to an embodiment of the present disclosure, the second linker peptide and the third linker peptide have 5 repeated amino acid sequences of GGGGS respectively. It is found by the inventor that while the second linker peptide and the third linker peptide have 5 repeated GGGGS respectively, the killing effect of the transgenic lymphocyte according to the embodiment of the present disclosure is stronger, and after being introduced into a body, the amount of the cytokines secreted by the body is lower, and the safety is higher. According to an embodiment of the present disclosure, the extracellular region has the amino acid sequence shown in SEQ ID NO: 1˜3.

In a fifth aspect of the present disclosure, the present disclosure provides a construct. According to an embodiment of the present disclosure, the construct includes a nucleic acid molecule, the nucleic acid molecule encodes a chimeric antigen receptor, and the chimeric antigen receptor is as defined previously. The construct according to the embodiment of the present disclosure is introduced into a receptor cell-T lymphocyte, and the previously described transgenic lymphocyte may be obtained. The transgenic lymphocyte obtained may specifically kill the single/double positive tumor cells of the first antigen and the second antigen, and has the longer and more powerful effect of killing and eliminating the tumor cells in vivo compared with the existing technology.

According to an embodiment of the present disclosure, the above construct may further include at least one of the following additional technical features.

According to an embodiment of the present disclosure, the construct further includes: a promoter, herein the promoter is operably connected with the nucleic acid molecule.

According to an embodiment of the present disclosure, the promoter is U6, H1, CMV, EF-1, LTR or RSV promoter.

According to an embodiment of the present disclosure, the vector of the construct is a non-pathogenic virus vector.

According to an embodiment of the present disclosure, the virus vector includes at least one selected from a retrovirus vector, a lentivirus vector and an adenovirus associated virus vector.

In a sixth aspect of the present disclosure, the present disclosure provides a method for preparing the previously described T lymphocyte or the previously described transgenic lymphocyte. According to the method of the embodiment of the present disclosure, the method includes: introducing the previously described construct or the previously described lentivirus into a lymphocyte or a T lymphocyte. The T lymphocyte or the transgenic lymphocyte prepared according to the method of the embodiment of the present disclosure may specifically kill the single/double positive tumor cells of the first antigen and the second antigen, and has the longer and more powerful effect of killing and eliminating the tumor cells in vivo compared with the existing technology.

In a seventh aspect of the present disclosure, the present disclosure provides a therapeutic composition for treating a cancer. According to an embodiment of the present disclosure, the therapeutic composition includes the previously described construct, the previously described lentivirus, the previously described T lymphocyte or the previously described transgenic lymphocyte. The pharmaceutical composition according to the embodiment of the present disclosure has the longer and more powerful effect of killing and eliminating the tumor cells in vivo.

According to an embodiment of the present disclosure, the cancer includes at least one selected from B lymphocyte leukemia and B cell lymphoma. The pharmaceutical composition according to the embodiment of the present disclosure may be applied to an immunotherapy for a patient with the B lymphocyte leukemia and the B cell lymphoma; for the patient with the B lymphocyte leukemia and the B cell lymphoma, the immunocyte therapy effect according to the embodiment of the present disclosure is superior to an existing treatment means; in addition, the dual CART cells in the present disclosure greatly reduce the possibility of recurrence of the patient with the B lymphocyte leukemia and the B cell lymphoma, and play a very significant role in promoting the treatment of the B lymphocyte leukemia and the B cell lymphoma.

In an eighth aspect of the present disclosure, the present disclosure provides an application of the previously described T lymphocyte, the previously described lentivirus, the previously described transgenic lymphocyte, the previously described construct or the previously described therapeutic composition in preparation of a drug for treating a cancer.

According to an embodiment of the present disclosure, the cancer includes at least one selected from the B lymphocyte leukemia and the B cell lymphoma. The T lymphocyte, the lentivirus, the transgenic lymphocyte, the construct or the therapeutic composition according to the embodiments of the present disclosure may be used in immunotherapy for the patient with the B lymphocyte leukemia and the B cell lymphoma as components of the drug for treating the cancer.

In a ninth aspect of the present disclosure, the present disclosure provides a method for treating a cancer. According to an embodiment of the present disclosure, the method includes administering at least one of the followings to a subject suffering from the cancer:
the previously described T lymphocyte;
the T lymphocyte introduced into the previously described lentivirus;
the previously described transgenic lymphocyte;
the T lymphocyte introduced into the previously described construct; and
the previously described therapeutic composition.

According to an embodiment of the present disclosure, the cancer includes at least one selected from the B lymphocyte leukemia and the B cell lymphoma.

In a tenth aspect of the present disclosure, the present disclosure provides an application of the previously described T lymphocyte, the previously described lentivirus, the previously described transgenic lymphocyte, the previously described construct or the previously described therapeutic composition in treatment of a cancer.

According to an embodiment of the present disclosure, the cancer includes at least one selected from the B lymphocyte leukemia and the B cell lymphoma.

In an eleventh aspect of the present disclosure, the present disclosure provides a method for improving lymphocyte activity. According to an embodiment of the present disclosure, the method includes: making the lymphocyte express a chimeric antigen receptor, herein the chimeric antigen receptor is as defined previously, and the lymphocyte activity includes at least one of the viability of the lymphocyte in a tumor patient and the killing ability of the lymphocyte in the tumor patient.

The additional aspects and advantages of the present disclosure are partially given in the following descriptions, and some may become apparent from the following descriptions, or understood from the practice of the present disclosure.

### BRIEF DESCRIPTION OF THE DRAWINGS

The above and/or additional aspects and advantages of the present disclosure may become apparent and easily understood from descriptions of embodiments in combination with the following drawings, herein:

FIG. 1 is a molecular structure diagram of Car-pCDHF32 according to an embodiment of the present disclosure.

FIG. 2 is a molecular structure diagram of Car-pCDHF34 according to an embodiment of the present disclosure.

FIG. 3 is a plasmid diagram of Car-pCDHF32 and Car-pCDHF34 according to an embodiment of the present disclosure.

FIG. 4 is a cell working principle diagram of pCDHF-32 according to an embodiment of the present disclosure.

FIG. 5 is a result diagram of positive rates of a flow cytometry of Car-pCDHF-32 and 34 according to an embodiment of the present disclosure.

FIG. 6 is a peak detection diagram of CD19 and CD22 antigen expression on the surface of Nalm6 and Raji cells according to an embodiment of the present disclosure.

FIG. 7 is an in vitro killing function diagram of Car-pCDHF32 and Car-pCDHF34 according to an embodiment of the present disclosure, herein,

A is a killing effect of Car-pCDHF32 and Car-pCDHF34 on K562/K562-CD19/K562-CD22/Nalm6/Raji cells;

B is a killing effect comparison of Car-pCDHF32 and Car-pCDHF34 on K562/K562-CD19/K562-CD22/Raji cells while the effect target ratio is 5:1; and

C is a cytokine secretion comparison of Car-pCDHF32 and Car-pCDHF34 in a K562/K562-CD19/K562-CD22/Raji cell killing experiment while the effect target ratio is 5:1.

FIG. 8 is a cytokine secretion diagram in the K562/K562-CD19/K562-CD22/Raji cell killing experiment by Car-pCDHF32 and Car-pCDHF34 according to an embodiment of the present disclosure.

FIG. 9 is a comparison diagram of an in vitro tumor killing effect between Car-pCDHF32 and single targets CarT and Loop 6 Car according to an embodiment of the disclosure.

FIG. 10 is a molecular structure diagram of pCDHF-60 according to an embodiment of the present disclosure.

FIG. 11 is a result diagram of positive rates, detected by fluorescent antigen staining, of CarT-pCDHF32 and CarT-pCDHF60 obtained after lentivirus packaging and infecting T cells according to an embodiment of the present disclosure.

FIG. 12 is a result diagram of Car-pCDHF32 and Car-pCDHF60 in vitro killing functions according to an embodiment of the present disclosure, herein,

A is a killing effect of Car-pCDHF32 and Car-pCDHF60 on K562/Nalm6 cells; and

B is a cytokine secretion condition in a K562/Nalm6 cell killing experiment by Car-pCDHF32 and Car-pCDHF60.

FIG. 13 is a schematic diagram of an in vivo function comparison experiment of CarT-pCDHF32 and CarT-pCDH F60 according to an embodiment of the present disclosure.

FIG. 14 is a result of the in vivo function comparison experiment of CarT-pCDHF32 and CarT-pCDHF60 according to an embodiment of the disclosure, herein, A is monitoring data of a mouse weight change of the Car-pCDHF32 and Car-pCDHF60 in vivo experiment; and

B is a mouse survival curve of the Car-pCDHF32 and Car-pCDHF60 in vivo experiment.

FIG. 15 is a molecular structure of Car-pCDHF31 according to an embodiment of the present disclosure.

FIG. 16 is a killing effect diagram of Car-pCDHF31 and Car-pCDHF60 on K562/K562-CD19/K562-BCMA/Daudi cells according to an embodiment of the present disclosure.

FIG. 17 is a cytokine secretion condition in a killing experiment of Car-pCDHF31 on the K562/K562-CD19/K562-BCMA/Daudi cells according to an embodiment of the present disclosure.

FIG. 18 is an in vitro killing effect of second and third linker peptides having 3˜6 repeated GGGGS in a dual specific CART structure according to an embodiment of the present disclosure.

FIG. 19 is a cytokine detection of the second and third linker peptides having 3˜6 repeated GGGGS in the dual specific CART structure according to an embodiment of the present disclosure.

In the drawings, T cell/T-cell refers to a T cell; Medium refers to a culture medium; Linker refers to a linker peptide; and Linker annotation refers to linker peptide annotation.

### DETAILED DESCRIPTION OF THE EMBODIMENTS

Embodiments of the present disclosure are described in detail below. The embodiments described below are exemplary, and are only used to explain the present disclosure, but may not be understood as limitation to the present disclosure. If the specific technologies or conditions are not indicated in the embodiments, it is performed according to the technologies or conditions described in documents in this field or according to product specifications. Reagents or instruments used that do not indicate manufacturers are conventional products that may be purchased in the market.

In addition, terms “first” and “second” are only used for a descriptive purpose, and may not be understood as indicating or implying relative importance or implicitly indicating the number of technical features indicated. Thus, the features defined as the “first” and “second” may explicitly or implicitly include at least one such feature. In the description of the present disclosure, “multiple” means at least two, such as two, and three, unless otherwise specifically defined.

A term “optionally” is used only for the descriptive purpose, and may not be understood as indicating or implying the relative importance. Thus, the feature defined as the “optionally” may explicitly or implicitly include or exclude this feature.

A single-chain antibody (scFv) is a genetic engineering antibody, herein a heavy chain variable region (VH) and a light chain variable region (VL) are linked with a flexible peptide linker. Compared with a Fab region of the whole antibody, the single-chain antibody shows the better tissue penetration pharmacokinetics, and has the complete antigen binding specificity because the antigen binding surface is not changed.

An immune costimulatory molecule refers to a cell surface molecule and its ligand that provide a costimulatory signal for the complete activation of the T lymphocyte or B lymphocyte, such as 4-1 BB, OX-40, CD40L, CD27, CD30, CD28, CD3 and their derivatives.

According to a specific embodiment of the present disclosure, the construction of one lentivirus vector is taken as an example. The inventor inserts a target nucleic acid into a virus genome at the position of some virus sequences in order to construct one lentivirus vector, thereby replication defective viruses are generated. In order to generate a virion, the inventor further constructs a packaging cell line (including gag, pol and env genes, but excluding a long terminal repeat (LTR) and a packaging component). The inventor introduces a recombinant plasmid containing the target gene into the packaging cell line together with lentivirus LTR and packaging sequence. The packaging sequence allows a recombinant plasmid RNA transcript to be packaged into a virus particle, and then secreted into a culture medium. The inventor then collects a matrix containing the recombinant lentivirus, selectively concentrates it, and uses it for gene transfer. The lentivirus vector may infect many cell types, including a dividing cell and a non-dividing cell.

In addition, according to an embodiment of the present disclosure, the lentivirus in the embodiment of the present disclosure is a compound lentivirus. In addition to common lentivirus genes gag, pol and env, it also contains other genes with regulatory and structural functions. The lentivirus vector is well-known to those skilled in the art, and the lentivirus includes a human immunodeficiency virus HIV-1 and HIV-2 and a simian immunodeficiency virus SIV. The lentivirus vector is generated by multiple attenuation of a HIV pathogenic gene, for example, deletion of all genes env, vif, vpr, vpu and nef makes the lentivirus vector form a biosafety vector. The recombinant lentivirus vector may infect the non-dividing cell, and may be used for gene transfer and nucleic acid sequence expression in vivo and in vitro at the same time. For example, in a suitable host cell, two or more vectors with packaging functions (gag, pol, env, rev and tat) may infect the non-dividing cell together. The targeting property of the recombinant virus is achieved by the combination of an antibody or a specific ligand (targeting a specific cell type receptor) with a membrane protein. At the same time, the targeting property of the recombinant virus makes the vector have a specific target by inserting an effective sequence (including a regulatory region) into the virus vector, together with another gene encoding a ligand of a receptor on a specific target cell. Various useful lentivirus vectors, as well as vectors generated by various methods and operations, are used to change the expression of cells.

According to an embodiment of the present disclosure, an adeno-associated virus vector (AAV) in the embodiment of the present disclosure may be constructed by using a deoxyribonucleic acid (DNA) of one or more well-known serum type adeno-associated virus vectors. In addition, according to an embodiment of the present disclosure, the embodiment of the present disclosure also includes a microgene. The microgene means that combinations (a selected nucleotide sequence and an operable relevant linkage sequence) are used to guide the expression of transformation, transcription and/or gene products in the host cell in vivo or in vitro. The “operable linkage” sequence includes an expression control sequence of a continuous target gene and an expression control sequence acting on a trans or remote control target gene.

In addition, the vector in the embodiment of the present disclosure also includes a conventional control element in cell transfection with the plasmid vector or/and cell infection with the virus vector. A large number of the expression control sequences (including natural, inducible and/or tissue specific promoters) may be used. According to an embodiment of the present disclosure, the promoter is an RNA polymerase promoter selected from U6, H1, pol I, pol II and pol III. According to an embodiment of the present disclosure, the promoter is a tissue specific promoter. According to an embodiment of the present disclosure, the promoter is an inducible promoter. According to an embodiment of the present disclosure, the promoter is a promoter selected from the selected vectors. According to an embodiment of the disclosure, while the lentivirus vector is selected, the promoter is U6, H1, CMV IE gene, EF-1α, ubiquitin C or glycerophosphate kinase (PGK) promoter. Other conventional expression control sequences include an optional marker or a reporter gene, including a nucleotide sequence encoding geneticin, hygromycin, ampicillin or puromycin drug resistance and the like. Other components of the vector include a replication starting point.

Technologies for constructing the vector are well-known to those skilled in the art, and these technologies include a conventional cloning technology.

According to an embodiment of the present disclosure, the composition in the embodiment of the present disclosure provided to a patient is better applied to biocompatible solution or an acceptable pharmaceutical carrier. Various therapeutic compositions as preparations are suspended or dissolved in pharmaceutically or physiologically acceptable carriers, such as normal saline; and isotonic salt solution or other more apparent formulas of people who are proficient in this way. The appropriate carrier depends to a large extent on the route of administration. Other isotonic sterile injections with and without water and sterile suspensions with and without water are pharmaceutically acceptable carriers.

These methods that express the specific chimeric antigen receptor targeting the antigens CD19 and CD22 are a part of combination therapy. These virus vectors and anti-tumor T cells for adoptive immunotherapy may be performed alone or in combination with other cancer treatment methods. Under appropriate conditions, one treatment method includes use of one or more drug therapies.

Schemes of the present disclosure are explained below in combination with the embodiments.

Those skilled in the art may understand that the following embodiments are only used to describe the present disclosure, and should not be considered as limiting the scope of the present disclosure. If the specific technologies or conditions are not indicated in the embodiments, it is performed according to the technologies or conditions described in documents in this field (for example, referring to “Guide to Molecular Cloning Experiment” written by J. Sambrook et al. and translated by Huang Peitang et. al., the third edition, Science Press) or according to product specifications. Reagents or instruments used that do not indicate manufacturers are conventional products that may be purchased in the market.

The present disclosure obtains a novel double Car structure for the CD19 and CD22 targets by optimizing the order of the light and heavy chains of tandem CD19 and CD22 single-chain antibody sequences and the length of the mutually connected linker peptides thereof. According to an embodiment of the present disclosure, the present application may prepare a CarT cell with a high positive rate, and has the high specificity and good killing effect on CD19 and CD22 target cells. In addition, this structure may be used to construct double target CarT with other targets, and has good universality.

### Embodiment 1: Sequence Selection of Single-Chain Antibody (scFv) Targeting CD19 and CD22

A B lymphocyte antigen CD19, also known as CD19, is a one-way type-I membrane protein that contains two Ig-like C2-type (immunoglobulin like) domains. The scFv sequence targeting CD19 selects an antibody FMC63-mlgG2a, FMC63-mlgG2a is a mouse-derived antibody against CD19 obtained by animal immunity in the last century, and its binding epitope is an Ig domain at a far-membrane end of CD19. FMC63-mlgG2ascFv is already successfully applied to an anti-CD19 CAR structure, such as Norvatis CTL019 and Juno Therapeutics JCAR015. The clinical trial of CTL019 and JCAR015 in B cell acute lymphoblastic leukemia achieves the better results, and the proportion of patients with complete remission is >70%. The variable area sequences of the light and heavy chains thereof are as follows:

Amino acid sequence of light chain variable region:

(SEQ ID NO: 25)
DIQMTQTTSSLSASLGDRVTISCRASQDISKYLNWYQQKPDGTVKLLIYHTSRLHSGVPSRFS
GSGSGTDYSLTISNLEQEDIATYFCQQGNTLPYTFGGGTKLEIT.

Amino acid sequence of heavy chain variable region:

(SEQ ID NO: 26)
EVKLQESGPGLVAPSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQGTSVTVSS.

CD22 is a type-I transmembrane glycoprotein and a member of the sialic acid binding immunoglobulin like lectin family. As an inhibitory co-receptor of a B cell receptor (BCR), CD22 has a negative regulation effect on a B cell activation signal, and the extracellular portion includes 7 immunoglobulin domains. The scFv sequence targeting CD22 selects an antibody M971, and its binding epitope is a CD22 near-membrane end Ig domain 5-7. M971-mlgG is a humanized antibody obtained by screening of an antibody library, and is widely used in CarT targeting CD22. For example, clinical data reported by Juno Therapeutics JCAR018 in 2017ASH shows that the proportion of patients with complete remission of RIR ALL is 78%. The variable area sequences of light and heavy chains thereof are as follows.

Amino acid sequence of light chain variable region:

(SEQ ID NO: 27)
DIQMTQSPSSLSASVGDRVTITCRASQTIWSYLNWYQQRPGKAPNLLIYAASSLQSGVPSRFS
GRGSGTDFTLTISSLQAEDFATYYCQQSYSIPQTFGQGTKLEIK.

Amino acid sequence of heavy chain variable region:

(SEQ ID NO: 28)
QVQLQQSGPGLVKPSQTLSLTCAISGDSVSSNSAAWNWIRQSPSRGLEWLGRTYYRSKWYN
DYAVSVKSRITINPDTSKNQFSLQLNSVTPEDTAVYYCAREVTGDLEDAFDIWGQGTMVTV.

### Embodiment 2: Construction of Double Specific CAR Plasmids (pCDHF-32, and 34) Targeting CD19 and CD22

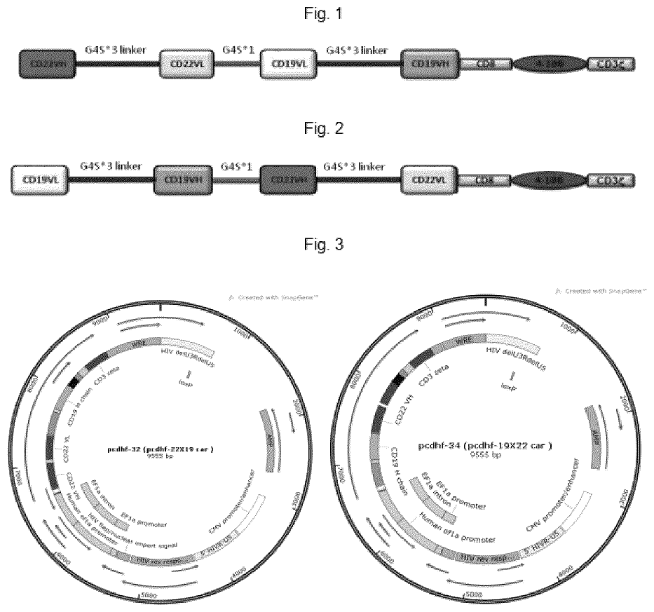

The molecular structure of Car-pCDHF32 is shown in FIG. 1.

The molecular structure of Car-pCDHF34 is shown in FIG. 2.

The plasmid diagram of Car-pCDHF32 and Car-pCDHF34 is shown in FIG. 3.

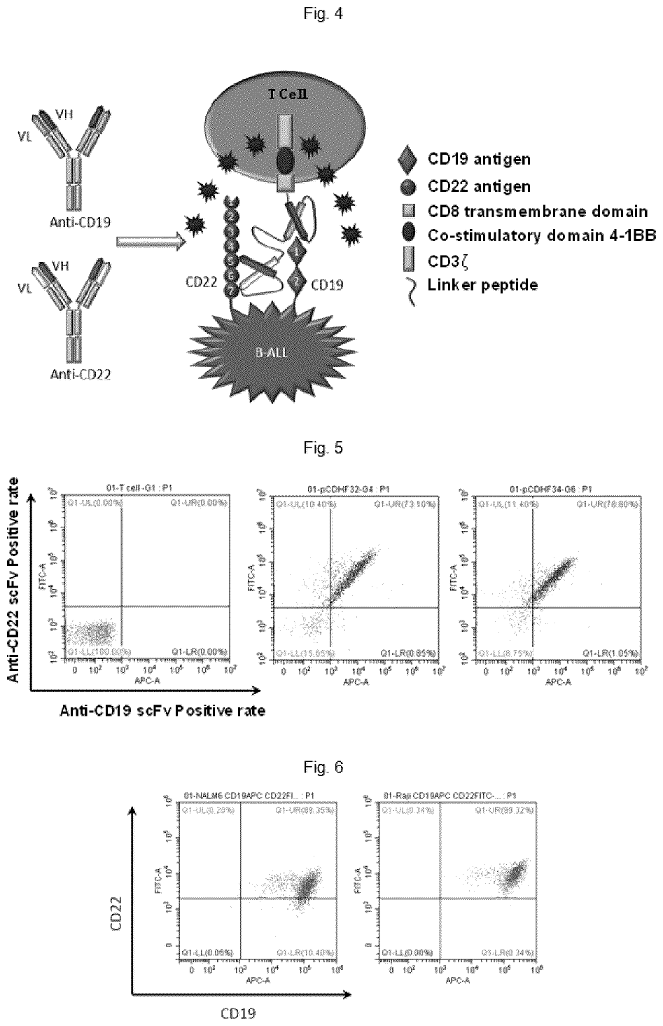

Herein, the cell working principle diagram of Car-pCDHF32 is shown in FIG. 4.

### Embodiment 3: Detection of Positive Rate of Double Specific CART (pCDHF-32, and 34) for CD19 and CD22

A lentivirus was packaged with a 293T cell, and the obtained lentivirus was infected with a T cell according to MOI=10:1 to prepare a CART cell (the positive rate of anti-CD19scFv was detected by second antibody APC Goat anti Mouse IgG (H+L), and the positive rate of anti-CD22scFv was detected by a self-produced fluorescent labeling antigen CD22-His-FITC), a flow cytometry was used to detect the titer of the lentivirus and a method for preparing the CART cell from the T cell infected with the lentivirus might be obtained from open channels. Results of the flow cytometry are shown in FIG. 5. In an upper left quadrant, the proportion of the cells shown is the positive rates of anti-CD19scFv and anti-CD22scFv, namely the positive rate of pCDHF-32/pCDHF-34. The flow cytometry results in the figure show that the positive rates of pCDHF-32 and pCDHF-34 are both more than 70%. This result shows that the double specific CART of this structure may prepare a cell product with the high positive rate.

### Embodiment 4: Verification of Double Specific CART (pCDHF-32, and 34) In Vitro Tumor Killing Function of CD19 and CD22

A Nalm6 cell is an acute lymphoblastic leukemia cell, and a Raji cell is a black Burkitt lymphoma cell. A flow cytometry was used to detect expression peaks of CD19 and CD22 antigens on the cell membrane surface thereof, and a specific methods was as follows: after 5E+05 cancer cells were taken and incubated with a flow antibody at 4° C. for 20 min (,detected the expression peak of the CD22 antigen on the cell surface with FITC anti-human CD22 (BD Pharmangen/555424), and detected the expression peak of the CD19 antigen on the cell surface with APC anti-human CD19 (Biolegend/302212)), then it was washed once with phosphate buffer solution (PBS), the cells were resuspended and detected by a flow cytometer. Results are shown in FIG. 6, and the Nalm6 cell is a CD19⁺/CD22⁺ cell line.

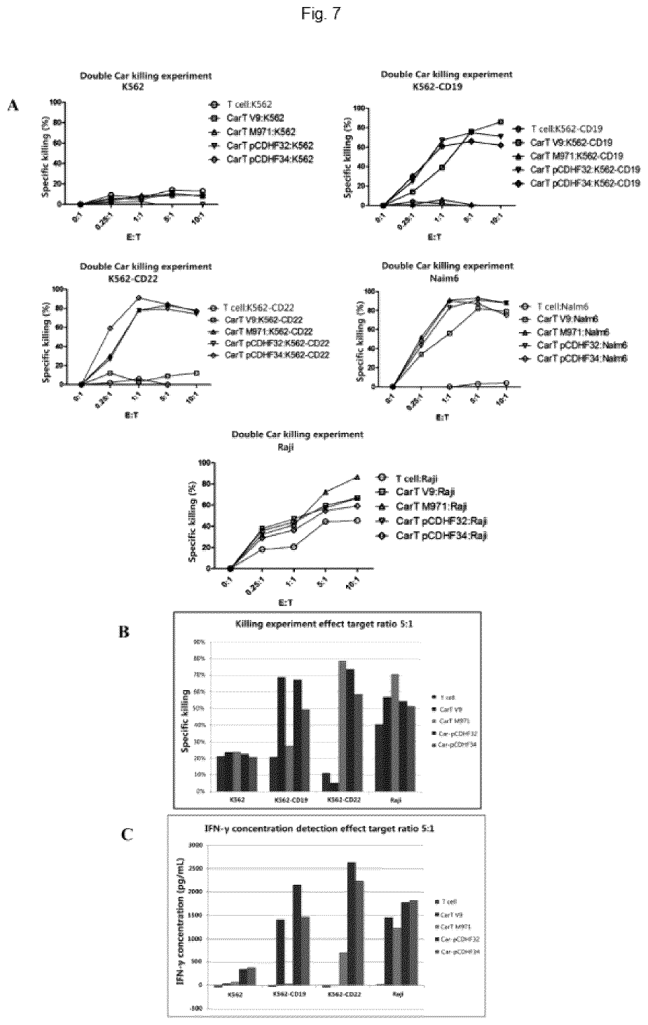

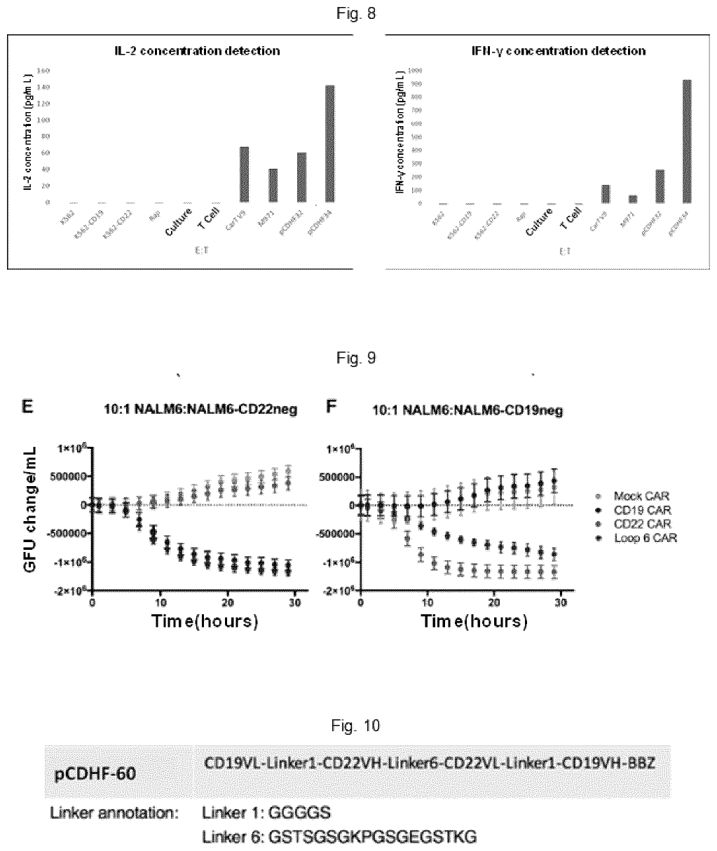

4 target cells, K562, K562-CD19, K562-CD22, and Nalm6 (or Raji) were each taken at 1E+07 cells, and cytocalceinTM violet 550 was firstly used to dye the target cells, 1×10E+05 cells/100 μL/well; effector cells (CarT V9/CarT M971/Car-pCDHF32/Car-pCDHF34, and the T cell was used as a control) and the target cells were added into a 96-well plate according to 0.25:1, 1:1, 5:1 and 10:1, the final volume was 200 μL, and after being co-cultured for 6 h, the cells were mixed uniformly and centrifuged, a supernatant was used to detect IL-2 and IFN-γ with a Human lL-2 and Human IFN gamma ELISA kit, a precipitation portion was resuspended with 100 μL of a binding buffer, it was centrifuged at 300 g for 5 min, 2.0 μL of APC-Annexin V and 1.5 μL of PI dye were added, it was incubated in the dark for 15 min, 100 μL of the binding buffer was added for resuspension, and a Beckmanc cou LTER flow cytometer was used to detect the apoptosis ratio of each target cell, as shown in FIG. 7. An enzyme-linked immunosorbent assay (ELISA) was used to detect IL-2 and IFN-γ in the supernatant of each well, and the concentrations are shown in FIG. 8, herein K562 is CD19 and CD22 negative cells, K562-CD19 and K562-CD22 are CD19 and CD22 single positive cells respectively and Raji and Nalm6 are both CD19 and CD22 double positive cells. Results show that Car-pCDHF32 and Car-pCDHF34 have the better killing effect on K562-CD19, K562-CD22, Raji and Nalm6′, and the killing effect of Car-pCDHF32 is still slightly better than that of Car-pCDHF34 while Car-pCDHF34 has the higher autocrine cytokines (shown in FIG. 7 and FIG. 8). It may be seen from the comparison of the in vitro tumor killing effects of Car-pCDHF32 and single target CarT that the killing effect of Car-pCDHF32 is close to'that of CD19 single target CarT, and this result is consistent with the in vitro tumor killing effect of Loop 6 Car (named pCDHF60 as follow-up control double Car in the present disclosure) with the optimal structure reported in a document “Preclinical Development of Bivalent Chimeric Antigen Receptors Targeting Both CD19 and CD22” (shown in FIG. 9).

### Embodiment 5: Comparison of Tumor Killing Functions In Vivo and In Vitro Between Double Specific CART (pCDHF-32) of CD19 and CD22 of Present Disclosure and Other Double Specific CART (pCDHF-60)

A double CarT plasmid (pCDHF-60) was constructed according to the optimal double CarT structure reported in the document “Preclinical Development of Bivalent Chimeric Antigen Receptors Targeting Both CD19 and CD22”, and its structure is shown in FIG. 10, which include Linker 6: GSTSGSGKPGSGEGSTKG (SECS ID NO: 24).

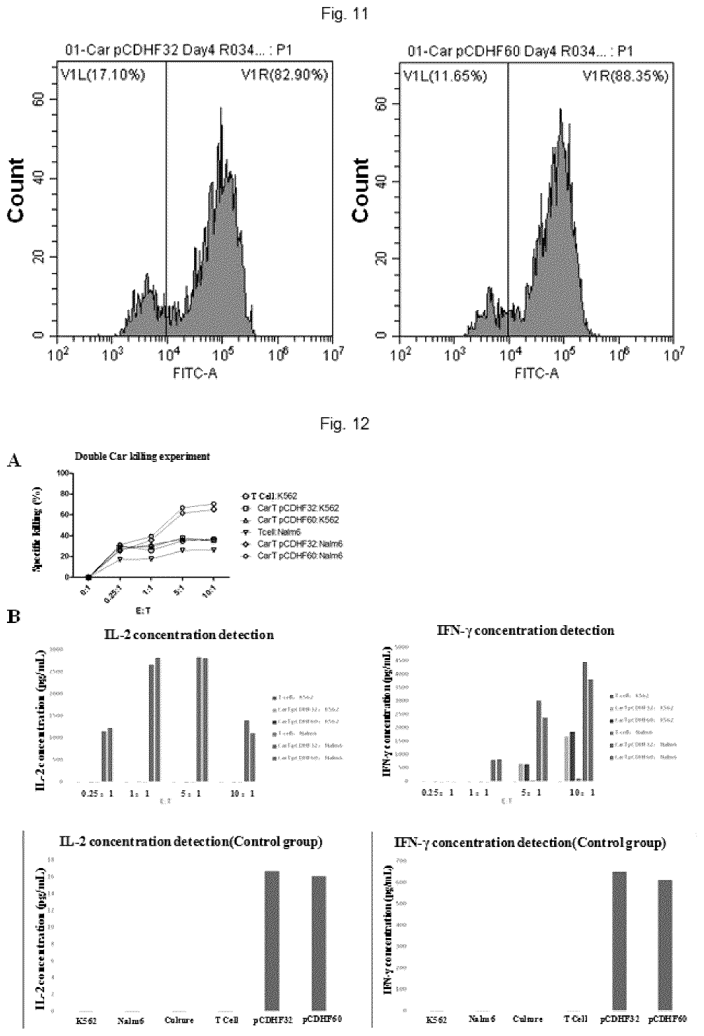

The positive rate of pCDHF60 detected by fluorescent antigen staining is shown in FIG. 11.

Nalm6 was used as the target cell, the in vitro killing abilities of pCDHF-32 and pCDHF-60 were compared with the method in Embodiment 4, and results are shown in FIG. 12.

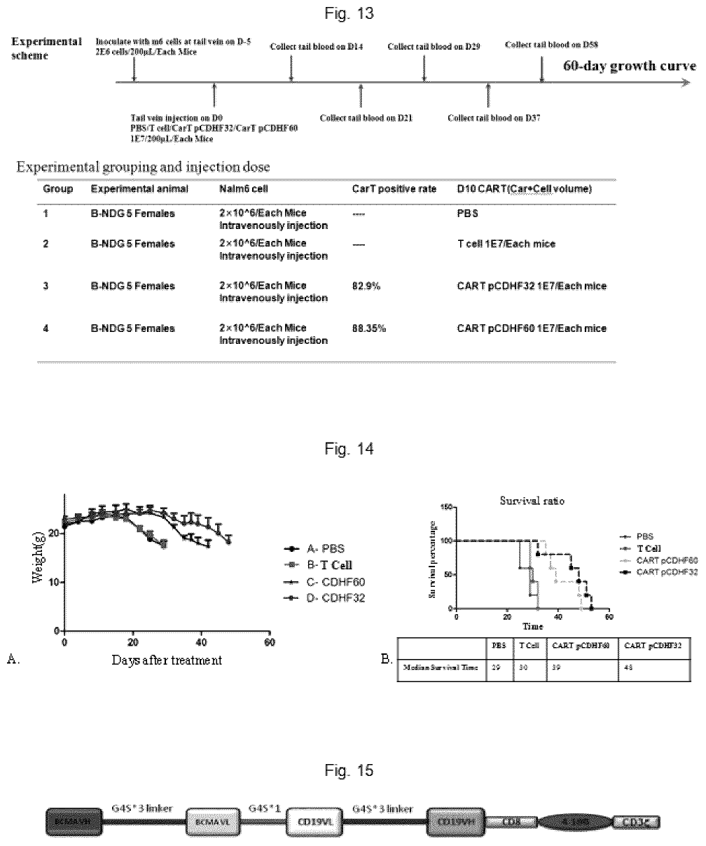

A NDG mouse was used to construct a mouse tumor model in a mode of tail vein injection using the Nalm6 cell as the target cell. According to the experimental scheme in FIG. 13, an in vivo function comparison experiment of CarT-pCDHF32 and CarT-pCDHF60 was performed. The mouse weight was recorded every other day during the experiment, the mouse survival curve was drawn (FIG. 14) and tail blood of the mouse is taken regularly. The proportion of tumor and CarT cells in the tail blood was monitored by the flow cytometry (Table 1).

TABLE 1

| | | (Blood, FACS)D21 | | (Blood, FACS)D29 | | (Blood, FACS)D36 | | (Blood, FACS)D43 | |
|---|---|---|---|---|---|---|---|---|---|
| Group | Animal ID | Nalm6 | T-Cell | Nalm6 | T-Cell | Nalm6 | T-Cell | Nalm6 | T-Cell |
| G1 PBS | 1-659 | 0.45% | 0.14% | 0.73% | 0.12% | | | | |
| | 2-667 | 2.04% | 0.06% | 16.98% | 0.00% | | | | |
| | 3-650 | 2.26% | 0.00% | | | | | | |
| | 4-653 | 3.08% | 0.04% | | | | | | |
| | 5-644 | 3.91% | 0.00% | | | | | | |
| Average | | 2.35% | 0.05% | 8.86% | 0.06% | | | | |
| G2 T Cell | 1-24 | 4.17% | 0.52% | 5.97% | 3.65% | | | | |
| | 2-665 | 1.45% | 0.64% | 0.83% | 6.07% | | | | |
| | 3-656 | 1.85% | 0.00% | 21.95% | 0.02% | | | | |
| | 4-651 | 2.12% | 0.10% | 4.27% | 0.45% | | | | |
| | 5-652 | 2.06% | 0.15% | 12.86% | 0.13% | | | | |
| Average | | 2.33% | 0.28% | 9.18% | 2.06% | | | | |
| G6 CDHF60 | 1-664 | 0.12% | 0.32% | 0.00% | 0.45% | 0.25% | 1.76% | 3.84% | 2.02% |
| | 2-654 | 0.03% | 0.21% | 0.02% | 0.08% | 0.38% | 0.27% | | |
| | 3-645 | 0.00% | 0.14% | 0.00% | 0.02% | 0.24% | 0.09% | 0.18% | 0.22% |
| | 4-663 | 0.04% | 0.37% | 0.03% | 0.13% | 0.07% | 0.04% | 0.11% | 0.18% |
| | 5-670 | 0.04% | 0.36% | 0.00% | 0.08% | 0.27% | 0.04% | 0.33% | 0.00% |
| Average | | 0.05% | 0.28% | 0.01% | 0.15% | 0.24% | 0.44% | 1.12% | 0.61% |
| G7 CDHF32 | 1-646 | 0.00% | 0.00% | 0.00% | 0.00% | 0.31% | 0.07% | | |
| | 2-680 | 0.00% | 1.07% | 0.00% | 0.00% | 0.02% | 0.02% | 0.08% | 0.02% |
| | 3-647 | 0.04% | 0.13% | 0.00% | 0.00% | 0.09% | 0.05% | 2.43% | 0.06% |
| | 4-655 | 0.08% | 0.15% | 0.00% | 0.00% | 0.03% | 0.00% | 0.43% | 0.00% |
| | 5-643 | 0.04% | 0.19% | 0.00% | 0.00% | 0.06% | 0.09% | 0.03% | 0.00% |
| Average | | 0.03% | 0.31% | 0.00% | 0.00% | 0.10% | 0.05% | 0.74% | 0.02% |

It may be seen from the above experimental data that although the in vitro killing experimental results show that the tumor killing efficiency of Car-pCDHF32 is slightly lower than that of Car-pCDHF60, the in vivo function experiment shows that Car-pCDHF32 has the longer CarT endurance and better effect of eliminating the blood tumor cells in vivo compared with Car-PCDH F60.

### Embodiment 6: Applicability of Double Specific CART Structure of Present Disclosure in Combination with Other Targets (pCDHF-31)

In order to verify whether the linker structure in the present disclosure may be used for other targets, CD19 and BCMA double target CarT was constructed with the linker structure in the present disclosure, herein the anti-BCMA scFv sequence portion used the light and heavy chain portions of a C11 D5.3 antibody, and the light and heavy chain variable region sequences thereof are as follows.

Amino acid sequence of light chain variable region:

(SEQ ID NO: 29)
DIVLTQSPASLAMSLGKRATISCRASESVSVIGAHLIHWYQQKPGQPPKLLIYLASNLETGVPA
RFSGSGSGTDFTLTIDPVEEDDVAIYSCLQSRIFPRTFGGGTKLEIK.

Amino acid sequence of heavy chain variable region:

(SEQ ID NO: 30)
QIQLVQSGPELKKPGETVKISCKASGYTFTDYSINWWKRAPGKGLKWMGWINTETREPAYAYD
FRGRFAFSLETSASTAYLQINNLKYEDTATYFCALDYSYAMDYWGQGTSVTVSS.

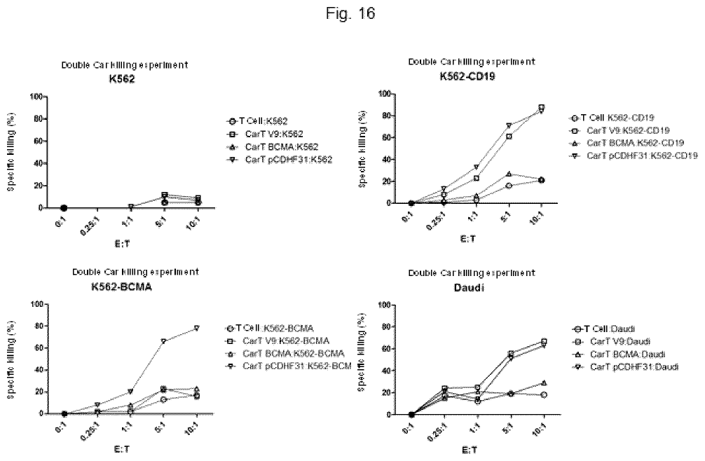

The molecular structure of Car-pCDHF31 is shown in FIG. 15.

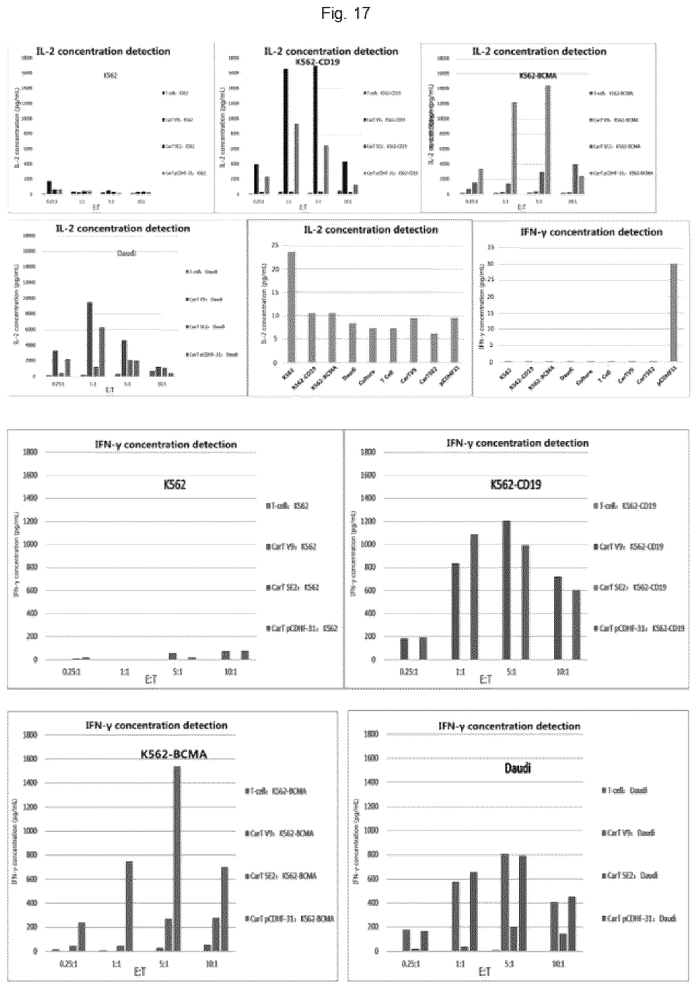

A Daudi cell is a human Burkitt's lymphoma cell (CD19+IBCMA+), K5621K562-CD191K562-BCMA/Daudi cells are used as the target cells, and in vitro killing activity results of CarT-pCDHF31 verified by the method in Embodiment 4 are shown in FIG. 16 and FIG. 17.

It may be seen from the above experimental data that after the target is replaced, the double CarT using the linker structure in the present disclosure still has the good functional activity. Therefore, it may be proved that the double Car with the linker structure in the present disclosure has the universality and universality of target replaceable.

### Embodiment 7: Influence of Length of Long Linker in Double Specific CART Structure of Present Disclosure on In Vitro Killing Effect

In order to verify whether the structure length of the linker between CD19VH and CD19VL and the linker between CD22VH and CD22VL in the present disclosure has an influence on the killing activity, in the present disclosure, 2˜6 repeated amino acid sequences of GGGGS were constructed respectively for the linker length between CD19VH and VL and the linker length between CD22VH and VL, and the in-vitro function verification was performed.

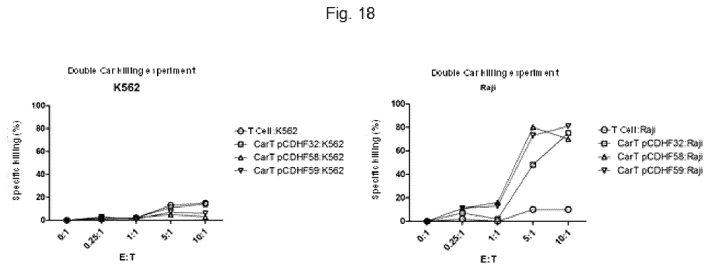

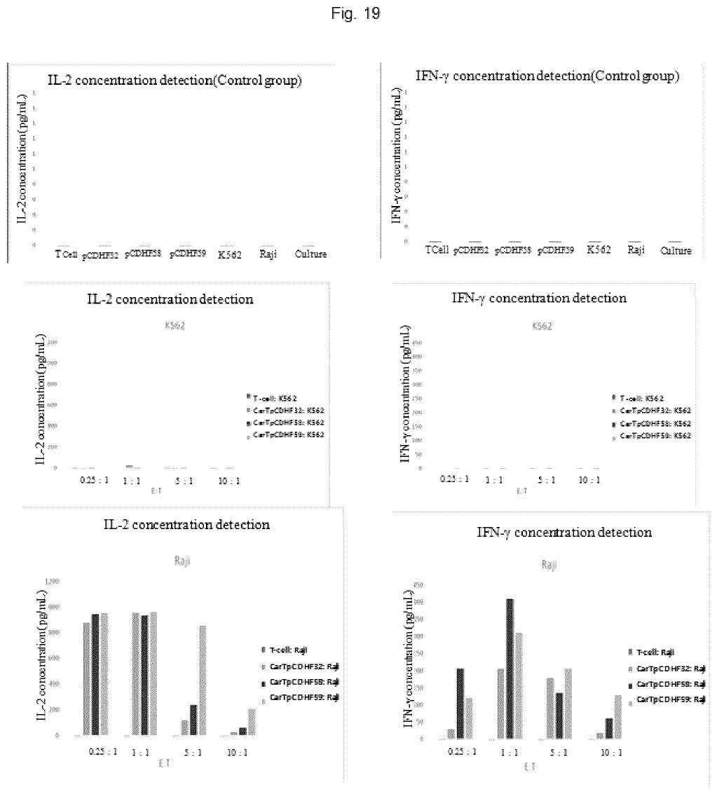

K562/Raji cells were used as the target cells, and the method in Embodiment 4 was used to verify the in vitro killing activity of double CART of the 2˜6 repeated amino acid sequences of GGGGS. FIG. 18 and FIG. 19 show some experimental results. Herein, CarT-pCDHF32 is double Car of which the second and third linker peptides have 3 repeated amino acid sequences of GGGGS, CarT-pCDHF58 is double Car of which the second and third linker peptides have 5 repeated amino acid sequences of GGGGS, and CarT-pCDHF59 is double Car of which the second and third linker peptides have 6 repeated amino acid sequences of GGGGS.

It may be seen from the above experimental data that, while E:T=1:1 and 5:1, CarT-pCDHF58 and CarT-pCDHF59 have the stronger in vitro killing effect than CarT-pCDHF32. However, the results of cytokine detection show that, while E:T=0.25:1 and 1:1, the secretion abilities of IL-2 of CarT-pCDHF32, CarT-pCDHF58 and CarT-pCDHF59 are basically equivalent; while E:T=5:1 and 10:1, the secretion of IL-2 of CarT-pCDHF59 is more. The secretion of IFN-r is higher in CarT-pCDHF58 and CarT-pCDHF59 than CarT-pCDHF32, except that while E:T=5:1, CarT-pCDHF58 is slightly lower than CarT-pCDHF32.

It may be seen from the experimental results of Embodiment 7 that the cytokine secretion amount caused by the double CART of which the second and third linker peptides have 3˜5 repeated amino acid sequences of GGGGS is less than the cytokine secretion amount caused by the double CART of which the second and third linker peptides have 6 repeated amino acid sequences of GGGGS, and the safety of the double CART of which the second and third linker peptides have 3˜5 repeated amino acid sequences of GGGGS is higher.

In descriptions of this description, descriptions of reference terms such as “one embodiment”, “some embodiments”, “examples”, “specific examples”, or “some examples” means that specific features, structures, materials, or characteristics described in combination with this embodiment or example are included in at least one embodiment or example of the present disclosure. In this description, the illustrative statements of the above terms unnecessarily refer to the same embodiments or examples. Furthermore, the specific features, structures, materials, or characteristics described may be combined in an appropriate mode in any one or more embodiments or examples. In addition, those skilled in the art may integrate and combine different embodiments or examples described in this description and the features of the different embodiments or examples in the case without conflicting.

Although the embodiments of the present disclosure are already shown and described above, it may be understood that the above embodiments are exemplary, and may not be understood as limitation to the present disclosure. Those of ordinary skill in the art may change, modify, replace and transform the above embodiments within the scope of the present disclosure.

What is claimed is:

1. A T lymphocyte, wherein the T lymphocyte expresses a chimeric antigen receptor, and the chimeric antigen receptor comprises:
an extracellular region, wherein the extracellular region comprises a first single-chain antibody, a second single-chain antibody, a first linker peptide and a CD8 hinge region, the first single-chain antibody specifically recognizes a first antigen, the second single-chain antibody specifically recognizes a second antigen, the first linker peptide is arranged between the first single-chain antibody and the second single-chain antibody,
the first single-chain antibody comprises a first heavy chain variable region, a first light chain variable region and a second linker peptide, the second linker peptide is arranged between the first heavy chain variable region and the first light chain variable region,
the second single-chain antibody comprises a second heavy chain variable region, a second light chain variable region and a third linker peptide, the third linker peptide is arranged between the second heavy chain variable region and the second light chain variable region,
the first linker peptide has one repeated amino acid sequence of GGGGS, and the second linker peptide and the third linker peptide independently have 2˜6 repeated amino acid sequences of GGGGS respectively;
a transmembrane region, wherein the transmembrane region is connected with the extracellular region, the transmembrane region comprises a transmembrane segment of CD8, and is embedded into a cell membrane of the T lymphocyte; and
an intracellular region, wherein the intracellular region is connected with the transmembrane region, and the intracellular region comprises an intracellular segment of 4-1 BB and a CD3 ζ chain.

2. The T lymphocyte according to claim 1, wherein the first single-chain antibody is a CD19 single-chain antibody, the second single-chain antibody is a CD22 single-chain antibody, the first antigen is CD19, the second antigen is CD22, the first heavy chain variable region is a CD19 heavy chain variable region, the first light chain variable region is a CD19 light chain variable region, the second heavy chain variable region is a CD22 heavy chain variable region, and the second light chain variable region is a CD22 light chain variable region; and
optionally, the first single-chain antibody is a CD19 single-chain antibody, the second single-chain antibody is a B cell maturation antigen (BCMA) single-chain antibody, the first antigen is CD19, the second antigen is BCMA, the first heavy chain variable region is a CD19 heavy chain variable region, the first light chain variable region is a CD19 light chain variable region, the second heavy chain variable region is a BCMA heavy chain variable region, and the second light chain variable region is a BCMA light chain variable region optionally, wherein the extracellular region has any one of the amino acid sequences shown in SEQ ID NOs: 1˜5.

3. The T lymphocyte according to claim 2, wherein the N terminal of the first linker peptide is connected with the C terminal of the second single-chain antibody, the C terminal of the first linker peptide is connected with the N terminal of the first single-chain antibody, and the C terminal of the first single-chain antibody is connected with the N terminal of the CD8 hinge region; and
optionally, the N terminal of the first linker peptide is connected with the C terminal of the CD22 single-chain antibody, the C terminal of the first linker peptide is connected with the N terminal of the CD19 single-chain antibody, and the C terminal of the CD19 single-chain antibody is connected with the N terminal of the CD8 hinge region;
optionally, wherein the N terminal of the third linker peptide is connected with the C terminal of the BCMA heavy chain variable region, the C terminal of the third linker peptide is connected with the N terminal of the BCMA light chain variable region, the C terminal of the BCMA light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region: and
optionally, the N terminal of the third linker peptide is connected with the C terminal of the CD22 heavy chain variable region, the C terminal of the third linker peptide is connected with the N terminal of the CD22 light chain variable region, the C terminal of the CD22 light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region.

4. The T lymphocyte according to claim 2, wherein the N terminal of the first linker peptide is connected with the C terminal of the CD19 single-chain antibody, the C terminal of the first linker peptide is connected with the N terminal of the CD22 single-chain antibody, and the C terminal of the CD22 single-chain antibody is connected with the N terminal of the CD8 hinge region;
optionally, wherein the N terminal of the second linker peptide is connected with the C terminal of the CD19 light chain variable region, the C terminal of the second linker peptide is connected with the N terminal of the CD19 heavy chain variable region, the C terminal of the CD19 heavy chain variable region is connected with the N terminal of the first linker peptide, the C terminal of the first linker peptide is connected with the N terminal of the CD22 heavy chain variable region, the N terminal of the third linker peptide is connected with the C terminal of the CD22 heavy chain variable region, and the C terminal of the third linker peptide is connected with the N terminal of the CD22 light chain variable region, and the C terminal of the CD22 light chain variable region is connected with the N terminal of the CD8 hinge region.

5. (canceled)

6. (canceled)

7. The T lymphocyte according to any ono of claims 1-6, wherein the second linker peptide and the third linker peptide independently have 3˜5 repeated amino acid sequences of GGGGS respectively, and preferably, the second linker peptide and the third linker peptide have 5 repeated amino acid sequences of GGGGS respectively.

8. (canceled)

9. A lentivirus, wherein the lentivirus carries a nucleic acid molecule encoding a chimeric antigen receptor, and the chimeric antigen receptor comprises:
an extracellular region, wherein the extracellular region comprises a first single-chain antibody, a second single-chain antibody, a first linker peptide and a CD8 hinge region, the first single-chain antibody specifically recognizes a first antigen, the second single-chain antibody specifically recognizes a second antigen, the first linker peptide is arranged between the first single-chain antibody and the second single-chain antibody,
the first single-chain antibody comprises a first heavy chain variable region, a first light chain variable region and a second linker peptide, the second linker peptide is arranged between the first heavy chain variable region and the first light chain variable region,
the second single-chain antibody comprises a second heavy chain variable region, a second light chain variable region and a third linker peptide, the third linker peptide is arranged between the second heavy chain variable region and the second light chain variable region,
the first linker peptide has one repeated amino acid sequence of GGGGS, and the second linker peptide and the third linker peptide independently have 2˜6 repeated amino acid sequences of GGGGS respectively;
a transmembrane region, wherein the transmembrane region is connected with the extracellular region, the transmembrane region comprises a transmembrane segment of CD8, and is embedded into a cell membrane of the T lymphocyte; and
an intracellular region, wherein the intracellular region is connected with the transmembrane region, and the intracellular region comprises an intracellular segment of 4-1 BB and a CD3 ζ chain.

10. The lentivirus according to claim 9, wherein the first single-chain antibody is a CD19 single-chain antibody, the second single-chain antibody is a CD22 single-chain antibody, the first antigen is CD19, the second antigen is CD22, the first heavy chain variable region is a CD19 heavy chain variable region, the first light chain variable region is a CD19 light chain variable region, the second heavy chain variable region is a CD22 heavy chain variable region, and the second light chain variable region is a CD22 light chain variable region; and
optionally, the first single-chain antibody is a CD19 single-chain antibody, the second single-chain antibody is a BCMA single-chain antibody, the first antigen is CD19, the second antigen is BCMA, the first heavy chain variable region is a CD19 heavy chain variable region, the first light chain variable region is a CD19 light chain variable region, the second heavy chain variable region is a BCMA heavy chain variable region, and the second light chain variable region is a BCMA light chain variable region;
optionally, wherein the N terminal of the third linker peptide is connected with the C terminal of the CD22 heavy chain variable region, the C terminal of the third linker peptide is connected with the N terminal of the CD22 light chain variable region, the C terminal of the CD22 light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region;
optionally, the N terminal of the second linker peptide is connected with the C terminal of the CD19 light chain variable region, the C terminal of the second linker peptide is connected with the N terminal of the CD19 heavy chain variable region, the C terminal of the CD19 heavy chain variable region is connected with the N terminal of the first linker peptide, the C terminal of the first linker peptide is connected with the N terminal of the CD22 heavy chain variable region, the N terminal of the third linker peptide is connected with the C terminal of the CD22 heavy chain variable region, the C terminal of the third linker peptide is connected with the N terminal of the CD22 light chain variable region, and the C terminal of the CD22 light chain variable region is connected with the N terminal of the CD8 hinge region;
optionally, the N terminal of the third linker peptide is connected with the C terminal of the BCMA heavy chain variable region, the C terminal of the third linker peptide is connected with the N terminal of the BCMA light chain variable region, the C terminal of the BCMA light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region;
optionally, the nucleic acid molecule encoding the extracellular region has any one of nucleotide sequences shown in SEQ ID NOs: 6˜10; optionally, the nucleic acid molecule encoding the transmembrane region has a nucleotide sequence shown in SEQ ID NO: 11; optionally, the nucleic acid molecule encoding the intracellular region has a nucleotide sequence shown in SEQ ID NO: 12; and
optionally, the nucleic acid molecule encoding the chimeric antigen receptor has any one of nucleotide sequences shown in SEQ ID NOs: 13˜17.

11. (canceled)

12. The lentivirus according to claim 10, wherein it carries a nucleic acid molecule with any one of nucleotide sequences shown in SEQ ID NOs: 18˜22.

13. A transgenic lymphocyte, wherein the lymphocyte expresses a chimeric antigen receptor, and the chimeric antigen receptor comprises:
an extracellular region, wherein the extracellular region comprises a first single-chain antibody, a second single-chain antibody, and a first linker peptide, the first single-chain antibody specifically recognizes a first antigen, the second single-chain antibody specifically recognizes a second antigen, the first linker peptide is arranged between the first single-chain antibody and the second single-chain antibody,
the first single-chain antibody comprises a first heavy chain variable region, a first light chain variable region and a second linker peptide, the second linker peptide is arranged between the first heavy chain variable region and the first light chain variable region,
the second single-chain antibody comprises a second heavy chain variable region, a second light chain variable region and a third linker peptide, the third linker peptide is arranged between the second heavy chain variable region and the second light chain variable region,
the first linker peptide has one repeated amino acid sequence of GGGGS, and the second linker peptide and the third linker peptide independently have 2˜6 repeated amino acid sequences of GGGGS respectively;
a transmembrane region, wherein the transmembrane region is connected with the extracellular region, and is embedded into a cell membrane of the lymphocyte; and
an intracellular region, wherein the intracellular region is connected with the transmembrane region, and the intracellular region comprises an intracellular segment of an immune co-stimulator molecule.

14. The transgenic lymphocyte according to claim 13, wherein the first single-chain antibody is a CD19 single-chain antibody, the second single-chain antibody is a CD22 single-chain antibody, the first antigen is CD19, the second antigen is CD22, the first heavy chain variable region is a CD19 heavy chain variable region, the first light chain variable region is a CD19 light chain variable region, the second heavy chain variable region is a CD22 heavy chain variable region, and the second light chain variable region is a CD22 light chain variable region; and
optionally, the first single-chain antibody is a CD19 single-chain antibody, the second single-chain antibody is a BCMA single-chain antibody, the first antigen is CD19, the second antigen is BCMA, the first heavy chain variable region is a CD19 heavy chain variable region, the first light chain variable region is a CD19 light chain variable region, the second heavy chain variable region is a BCMA heavy chain variable region, and the second light chain variable region is a BCMA light chain variable region.

15. The transgenic lymphocyte according to claim 13, wherein the intracellular segment of the immune co-stimulatory molecule is independently selected from at least one of 4-1 BB, OX-40, CD40L, CD27, CD30, CD28 and their derivatives;
optionally, the intracellular segment of the immune co-stimulatory molecule is an intracellular segment of 4-1 BB and CD3;
optionally, the lymphocyte is a CD3+ T lymphocyte;
optionally, the lymphocyte is a CD8+ T lymphocyte;
optionally, the lymphocyte is a natural killer cell; and
optionally, the lymphocyte is a natural killer T cell.

16. The transgenic lymphocyte according to claim 14, wherein the N terminal of the first linker peptide is connected with the C terminal of the second single-chain antibody, the C terminal of the first linker peptide is connected with the N terminal of the first single-chain antibody, and the C terminal of the first single-chain antibody is connected with the N terminal of the CD8 transmembrane region;
optionally, the N terminal of the first linker peptide is connected with the C terminal of the CD22 single-chain antibody, the C terminal of the first linker peptide is connected with the N terminal of the CD19 single-chain antibody, and the C terminal of the CD19 single-chain antibody is connected with the N terminal of the transmembrane region;
optionally, the N terminal of the first linker peptide is connected with the C terminal of the CD19 single-chain antibody, the C terminal of the first linker peptide is connected with the N terminal of the CD22 single-chain antibody, and the C terminal of the CD22 single-chain antibody is connected with the N terminal of the transmembrane region;
optionally, the N terminal of the third linker peptide is connected with the C terminal of the CD22 heavy chain variable region, the C terminal of the third linker peptide is connected with the N terminal of the CD22 light chain variable region, the C terminal of the CD22 light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the transmembrane region;
optionally, the N terminal of the second linker peptide is connected with the C terminal of the CD19 light chain variable region, the C terminal of the second linker peptide is connected with the N terminal of the CD19 heavy chain variable region, the C terminal of the CD19 heavy chain variable region is connected with the N terminal of the first linker peptide, the C terminal of the first linker peptide is connected with the N terminal of the CD22 heavy chain variable region, the N terminal of the third linker peptide is connected with the C terminal of the CD22 heavy chain variable region, and the C terminal of the third linker peptide is connected with the N terminal of the CD22 light chain variable region, and the C terminal of the CD22 light chain variable region is connected with the N terminal of the transmembrane region;
optionally, the N terminal of the third linker peptide is connected with the C terminal of the BCMA heavy chain variable region, the C terminal of the third linker peptide is connected with the N terminal of the BCMA light chain variable region, the C terminal of the BCMA light chain variable region is connected with the N terminal of the first linker peptide, the C terminal of the first linker peptide is connected with the N terminal of the CD19 light chain variable region, the N terminal of the second linker peptide is connected with the C terminal of the CD19 light chain variable region, the C terminal of the second linker peptide is connected with the N terminal of the CD19 heavy chain variable region, and the C terminal of the CD19 heavy chain variable region is connected with the N terminal of the CD8 hinge region;
optionally, the second linker peptide and the third linker peptide independently have 3˜5 repeated amino acid sequences of GGGGS respectively;
optionally, the second linker peptide and the third linker peptide have 5 repeated amino acid sequences of GGGGS respectively; and
optionally, the extracellular region has any one of amino acid sequences shown in SEQ ID NOs: 1˜5.

17. A construct, wherein the construct comprises a nucleic acid molecule, the nucleic acid molecule encodes a chimeric antigen receptor, and the chimeric antigen receptor is as defined according to claim 1.

18. The construct according to claim 17, further comprising: a promoter, wherein the promoter is operably connected with the nucleic acid molecule:
optionally, wherein the promoter is U6, H1, CMV, EF-1, LTR or RSV promoter;
optionally, wherein the vector of the construct is a non-pathogenic virus vector; and
optionally, the virus vector comprises at least one selected from a retrovirus vector, a lentivirus vector and an adenovirus associated virus vector.

19. (canceled)

20. (canceled)

21. A method for preparing the T lymphocyte according to claim 1, comprising:
introducing a construct into a lymphocyte or a T lymphocyte, wherein the construct comprises:
a nucleic acid molecule, the nucleic acid molecule encodes a chimeric antigen receptor, and the chimeric antigen receptor is as defined according to claim 1.

22. A therapeutic composition for treating a cancer, comprising:
the T lymphocyte according to claim 1.

23. The therapeutic composition according to claim 22, wherein the cancer comprises at least one selected from a B lymphocyte leukemia and a B cell lymphoma.

24. (canceled)

25. (canceled)

26. A method for treating a cancer, wherein the method comprises administering at least one of the followings to a subject suffering from the cancer:
the T lymphocyte according to claim 1; or
the therapeutic composition comprising the T lymphocyte according to claim 1.

27. The method according to claim 26, wherein the cancer comprises at least one selected from the B lymphocyte leukemia and the B cell lymphoma.

28. (canceled)

29. (canceled)

30. A method for improving lymphocyte activity, wherein the method comprises: making the lymphocyte express a chimeric antigen receptor, wherein the chimeric antigen receptor is as defined according to claim 1, and
the lymphocyte activity comprises at least one of the viability of the lymphocyte in a tumor patient and the killing ability of the lymphocyte in the tumor patient.
